(19) United States
(12) Patent Application Publication
(10) Pub. No.: US 2026/0306484 A1
(43) Pub. Date: Oct. 8, 2026

(54) INVERSE AGONISTS OF RAR RELATED ORPHAN RECEPTORS (RORS)

(71) Applicant: 11949098 CANADA INC. (NUREDCO) c/o Jean I. TCHERVENKOV, BROSSARD (CA)

(72) Inventors: Jean I. TCHERVENKOV, Brossard (CA); Ahmed FOUDA, Cote Saint Luc (CA); Steven PARASKEVAS, Montreal (CA); Sarita NEGI, Montreal (CA); Jonathan COOLS-LARTIGUE, Montreal (CA)

(21) Appl. No.: 19/482,319

(22) PCT Filed: May 8, 2024
(86) PCT No.: PCT/CA2024/050624

§ 371 Date: Nov. 7, 2025

Related U.S. Application Data
(60) Provisional application No. 63/464,994, filed on May 9, 2023.

(51) Int. Cl. A61K 31/496; A61K 31/167; A61K 31/381; A61K 31/4178; A61K 31/4365; A61K 31/44; A61K 31/454; A61K 31/4545; A61K 31/475; A61K 31/497; A61K 31/506; A61K 31/513; A61K 31/519; A61K 31/5377; A61K 31/7048; A61K 33/243; A61K 38/14; A61K 39/395; A61K 40/11; A61K 40/31; A61P 35/00; G01N 33/575

(52) U.S. Cl. CPC A61K 31/496; A61K 31/167; A61K 31/381; A61K 31/4178; A61K 31/4365; A61K 31/44; A61K 31/454; A61K 31/4545; A61K 31/475; A61K 31/497; A61K 31/506; A61K 31/513; A61K 31/519; A61K 31/5377; A61K 31/7048; A61K 33/243; A61K 38/14; A61K 39/3955; A61K 40/11; A61K 40/31; A61P 35/00; G01N 33/575

(57) ABSTRACT

The invention relates to the use of a ROR inverse agonist or a pharmaceutically acceptable salt thereof for the manufacture of a medicament for modulating RORs and/or controlling autoimmune diseases and antibody mediated rejection in a patient. The RORs mediated disease or autoimmune disease includes HIV, cancer, celiac disease, diabetes mellitus type 1, Graves' disease, inflammatory bowel disease, multiple sclerosis, psoriasis, rheumatoid arthritis, systemic lupus erythematosus, asthma, dermatitis, fatty liver disease, Crohn's disease, cardiovascular disease, inflammatory diseases, neurological disorder, multiple sclerosis, acute respiratory distress syndrome and arteriosclerosis.

### FIELD OF THE INVENTION

This invention relates to novel inverse agonists of retinoic-acid-receptor-related orphan nuclear receptors (RORs) alpha (RORα or RORA) and gamma (RORC, RORγ or RORγt) and their use in the control of autoimmune diseases, antibody mediated allograft rejection, and other associated diseases involving increased or decreased activity of RORα or RORγt or their controlled genes and gene products which include many types of cancers, metabolic and inflammatory disorders.

### BACKGROUND

An autoimmune disease is a condition arising from an abnormal immune response to a normal body part. There are at least many types of autoimmune diseases. Nearly any body part can be involved. Common symptoms include low grade fever and feeling tired. Often symptoms come and go.

The cause is generally unknown. Some autoimmune diseases such as lupus run in families, and certain cases may be triggered by infections or other environmental factors. Some common diseases that are generally considered autoimmune include celiac disease, diabetes mellitus type 1, Graves' disease, inflammatory bowel disease, multiple sclerosis, psoriasis, rheumatoid arthritis, and systemic lupus erythematosus. The diagnosis can be difficult to determine.

Treatment depends on the type and severity of the condition. Nonsteroidal anti-inflammatory drugs (NSAIDs) and immunosuppressants are often used. Intravenous immunoglobulin may also occasionally be used. While treatment usually improves symptoms, they do not typically cure the disease.

Chronic antibody injury is a serious threat to allograft outcomes and is therefore the center of active research. In the continuum of allograft rejection, the development of antibodies plays a critical role. In recent years, an increased recognition of molecular and histologic changes has provided a better understanding of antibody-mediated rejection (AMR), as well as potential therapeutic interventions. However, several pathways are still unknown, which accounts for the lack of efficacy of some of the currently available agents that are used to treat rejection.

The retinoic acid receptor-related (ROR) sub-family of orphan nuclear receptors was initially identified on the basis of sequence similarities to the retinoic acid and retinoid X receptor families. Through alternative promoter usage and exon splicing, the ROR genes encode different isoforms of RORα, β and γ, which exhibit differential tissue expression and functions. RORγt is a differentially spliced isoform of RORY, that differs only in the N-terminus by the presence of 21 additional amino acids in RORY. The endogenous physiological ligands for RORγt have recently been identified as 7β-27-dihydroxy cholesterol, and two other cholesterol biosynthetic intermediates.

RORγt is exclusively expressed in cells of the immune system including CD4⁺ CD8⁺ double positive thymocytes5, Th17, Tc17, and γδ T cells, as well as a subset of innate lymphoid cells (ILCs) and regulatory T cells (Tregs). RORγt is a key transcription factor driving Th17 cell differentiation, and production of IL-17A, IL-17F and IL-22 in innate and adaptive immune cells, also termed “type 17” cells. Th17 cytokines, IL-17A, IL-17F, and IL-22, stimulate tissue cells to produce a panel of inflammatory chemokines, cytokines and metalloproteases, resulting in the recruitment of granulocytes to sites of inflammation. The Th17 cell subset has been shown to be the major pathogenic population in several models of autoimmune inflammation, including collagen-induced arthritis (CIA) and experimental autoimmune encephalomyelitis (EAE). RORγt deficient mice show impaired Th17 cell differentiation in vitro, significantly reduced Th17 cell populations in vivo, and decreased susceptibility to EAE and intestinal inflammation. RORγt-deficient T cells fail to induce colitis in the mouse T cell transfer model.

Human genetic studies have shown association of polymorphisms in the genes for Th17 cell-surface receptors, IL-23R and CCR6, with susceptibility to inflammatory bowel disease (IBD), multiple sclerosis (MS), rheumatoid arthritis (RA) ankylosing spondylitis (AS) and psoriasis. Clinical modulation of the IL-23/IL-17 pathway through biologics targeting IL-12/23, IL-23, IL-17A or IL-17RA has provided validation of its critical role in human autoimmune diseases. RORγt is a nuclear receptor target in the IL-23/IL-17 pathway and has been shown to be tractable to modulation by oral small molecules. Indeed, other nuclear receptors have been successfully targeted by orally available small molecules that are now marketed drugs.

There is a need to have modulators that inhibit the activity of RORs to control autoimmune diseases and antibody mediated rejection. Other modulators can activate RORs and can be useful in the treatment of many types of lymphomas and cancer. A Modulator effect is a pharmacological effect that can be stimulatory or inhibitory. The modulators can be categorized as activators or inhibitors. They can be also categorized as agonists, antagonists, partial agonists and inverse agonists.

### SUMMARY

According to an embodiment, there is provided the use of a ROR inverse agonist or a pharmaceutically acceptable salt thereof for the manufacture of a medicament for modulating RORs and/or controlling autoimmune diseases and antibody mediated rejection in a patient.

According to an embodiment, there is provided a method of preventing or treating a RORs mediated disease, autoimmune disease or antibody mediated rejection in a patient in a patient in need thereof, which comprises administering to said patient a therapeutically effective amount of a ROR inverse agonist, its pharmaceutically acceptable salt or stereoisomers thereof.

The RORs mediated disease or autoimmune disease is HIV, cancer, celiac disease, diabetes mellitus type 1, Graves' disease, inflammatory bowel disease, multiple sclerosis, psoriasis, rheumatoid arthritis, systemic lupus erythematosus, asthma, dermatitis, fatty liver disease, Crohn's disease, cardiovascular disease, inflammatory diseases, neurological disorder, multiple sclerosis, acute respiratory distress syndrome and arteriosclerosis.

The cancer is prostate cancer, breast cancer, ovarian cancer, multiple myeloma, brain cancer, glioma, lung cancer, salivary cancer, stomach cancer, thymic epithelial cancer, thyroid cancer, leukemia, melanoma, lymphoma, gastric cancer, pancreatic cancer, kidney cancer, bladder cancer, colon cancer, gastroesophageal adenocarcinoma and liver cancer.

The inverse agonist of RORs is a compound of Formula (I), (II), (III), or (IV), a pharmaceutically acceptable salts thereof, or stereoisomers thereof:

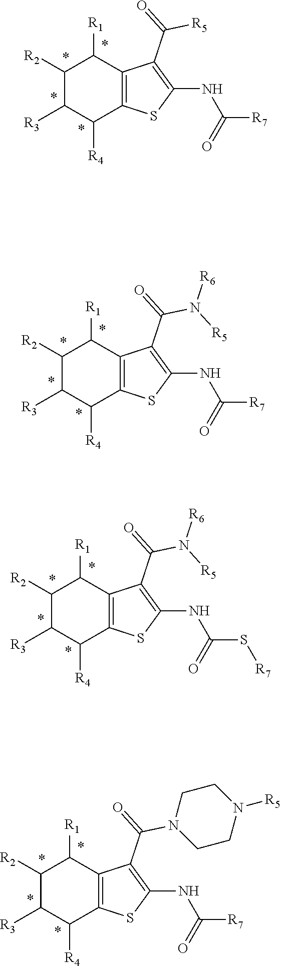

wherein,

R1, R2, R3, and/or R4 group(s) is/are H, halogen, NO2, 1-6 alkoxy, OH, NH2, 1-6 alkyl, 1-6 alkenyl, 1-6 haloalkyl, N-dialkyl, haloalkoxy, 1-6 hydroxyalkyl, and/or —CO2 (1-6 alkyl);

R5 is a substituted or unsubstituted five or six membered saturated or unsaturated heterocycle, aryl, alkylarene, halo aryl, ring substituted alkylarene, ring substituted alkylhexane, ring substituted alkylcyclopentane, haloaryl, benzene, phenyl, benzyl, pyridine, pyrimidine, pyridine, imidazole, diazole, triazole, thiadiazole, imidazolidine, thizolidine, pyrrolidine, piperazine, piperidine, pyridazine, pyrazine, triazine, 1H pyrrole, 2H pyrrole, pyrroline, pyrazolidine, pyrazoline, thiazole, isothiazole, isoxazole, haloalkyl, cyanoalkyl, methylpyrimidine, toluene, methylpyridine, methylimidazole, methyldiazole, methyltriazole, methylthiadiazole, methylimidazolidine, methylthizolidine, methylpyrrolidine, methylpiperazine, methylpiperidine, methylpyridazine, methylpyrazine, methyltriazine, methylpyrrole, methylpyrroline, methylpyrazolidine, methylpyrazoline, methylthiazole, methylisothiazole, methylisoxazole, or arylalkyl.

R6 is H, 1-6 alkyl, or may form a five or six ring structure with R5; and

R7 is a substituted or unsubstituted five or six membered saturated or unsaturated heterocycle, ring substituted alkylarene, ring substituted alkylhexane, ring substituted alkylcyclopentane, substituted haloaryl, substituted benzene, substituted phenyl, benzyl, pyrimidine, pyridine, imidazole, diazole, triazole, thiadiazole, imidazolidine, thizolidine, pyrrolidine, piperazine, aryl, halo aryl, alkylarene, piperidine, pyridazine, pyrazine, triazine, 1H pyrrole, 2H pyrrole, pyrroline, pyrazolidine, pyrazoline, thiazole, isothiazole, isoxazole, cyanoalkyl, methylpyrimidine, toluene, methylpyridine, methylimidazole, methyldiazole, methyltriazole, methylthiadiazole, methylimidazolidine, methylthizolidine, methylpyrrolidine, methylpiperazine, methylpiperidine, methylpyridazine, methylpyrazine, methyltriazine, methylpyrrole, methylpyrroline, methylpyrazolidine, methylpyrazoline, methylthiazole, methylisothiazole, methylisoxazole, or arylalkyl.

The preferred compound of Formula (I), (II), (III), or (IV) pharmaceutically acceptable salts thereof, or stereoisomers thereof is selected from the group consisting of:
N-(3-(4-benzylpiperazine-1-carbonyl)-4,5,6,7-tetrahydro-benzo[b]thiophen-2-yl)-2-fluorobenzamide;
N-(3-(benzylcarbamoyl)-4,5,6,7-tetrahydrobenzo[b]thiophen-2-yl)-5-chloro-2-(methylthio)pyrimidine-4-carboxamide;
5-chloro-N-(3-{[(1,1-dioxidotetrahydro-3-thienyl)amino]-carbonyl}-6-methyl-4,5,6,7-tetrahydro-1-benzothien-2-yl)-2-(methylthio)-4-pyrimidine-carboxamide;
N-(6-ethyl-3-{[(2-methylphenyl)amino]carbonyl}-4,5,6,7-tetrahydro-1-benzothien-2-yl)-1-methyl-1H-pyrazole-5-carboxamide;
N-{6-tert-butyl-3-[(4-methyl-1-piperazinyl) carbonyl]-4,5,6,7-tetrahydro-1-benzothien-2-yl}-2-fluorobenzamide;
2-fluoro-N-{6-methyl-3-[(4-methyl-1-piperazinyl) carbonyl]-4,5,6,7-tetrahydro-1-benzothien-2-yl}benzamide;
N-benzyl-2-[(trifluoroacetyl)amino]-4,5,6,7-tetrahydro-1-benzothiophene-3-carboxamide;
N-benzyl-2-({[(4-methyl-2-pyrimidinyl)thio]acetyl}amino)-4,5,6,7-tetrahydro-1-benzothiophene-3-carboxamide;
N-benzyl-2-[(1-piperidinylacetyl)amino]-4,5,6,7-tetrahydro-1-benzothiophene-3-carboxamide;
N-benzyl-2-{[(4-methyl-1-piperazinyl) acetyl]amino}-4,5,6,7-tetrahydro-1-benzothiophene-3-carboxamide;
N-benzyl-2-{[(4-methyl-1-piperidinyl) acetyl]amino}-4,5,6,7-tetrahydro-1-benzothiophene-3-carboxamide;
N-(3-(2-methylpyrrolidine-1-carbonyl)-4,5,6,7-tetrahydro-benzo[b]thiophen-2-yl) nicotinamide;
N-(3-((2R,4R)-2,4-dimethylpiperidine-1-carbonyl)-4,5,6,7-tetrahydrobenzo[b]thiophen-2-yl) nicotinamide;
N-(3-(((1r,4r)-4-hydroxycyclohexyl) carbamoyl)-4,5,6,7-tetrahydrobenzo[b]thiophen-2-yl) nicotinamide;
N-(3-(3-ethylpyrrolidine-1-carbonyl)-4,5,6,7-tetrahydro-benzo[b]thiophen-2-yl) nicotinamide;
N-benzyl-2-(2-((1-methyl-1H-imidazol-2-yl)thio) acetamido)-4,5,6,7-tetrahydrobenzo[b]thiophene-3-carboxamide;
N-(3-(3-(hydroxymethyl) pyrrolidine-1-carbonyl)-4,5,6,7-tetrahydrobenzo[b]thiophen-2-yl) nicotinamide;
N-(6,6-dimethyl-3-(morpholine-4-carbonyl)-4,5,6,7-tetrahydrobenzo[b]thiophen-2-yl) pyrazine-2-carboxamide;
N-(3-(((1-methyl-1H-1,2,4-triazol-3-yl)methyl) carbamoyl)-4,5,6,7-tetrahydrobenzo[b]thiophen-2-yl) nicotinamide;
(S)—N-(3-((1-cyanoethyl) carbamoyl)-4,5,6,7-tetrahydro-benzo[b]thiophen-2-yl) nicotinamide;
N-(3-((piperidin-4-ylmethyl) carbamoyl)-4,5,6,7-tetrahydro-benzo[b]thiophen-2-yl) nicotinamide;
N-(3-benzoyl-4,5,6,7-tetrahydrobenzo[b]thiophen-2-yl) pyrazine-2-carboxamide; and
N-(3-benzoyl-4,5,6,7-tetrahydrobenzo[b]thiophen-2-yl)-5-methyl-2-(methylsulfonyl)pyrimidine-4-carboxamide.

Another preferred compound, or a pharmaceutically acceptable salt thereof, is selected from the following compounds:

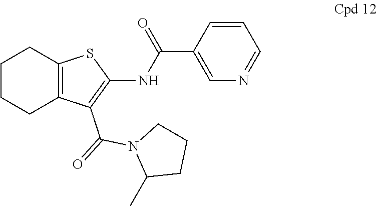 [SMILES: CC1CCCN1C(=O)c1c(NC(=O)c2cccnc2)sc2c1CCCC2]

N-(3-(2-methylpyrrolidine-1-carbonyl)-4,5,6,7-tetrahydrobenzo[b]thiophen-2-yl) nicotinamide

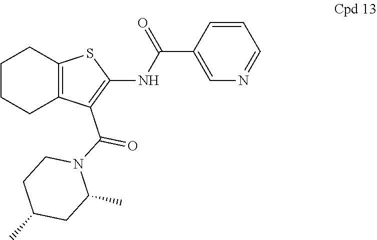 [SMILES: C[C@@H]1CCN(C(=O)c2c(NC(=O)c3cccnc3)sc3c2CCCC3)[C@H](C)C1]

N-(3-((2R,4R)-2,4-dimethylpiperidine-1-carbonyl)-4,5,6,7-tetrahydrobenzo[b]thiophen-2-yl) nicotinamide

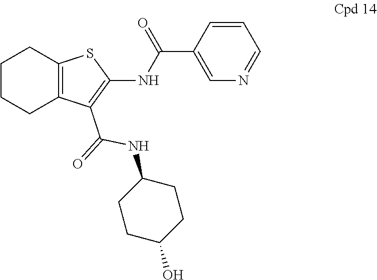 [SMILES: O=C(Nc1sc2c(c1C(=O)N[C@H]1CC[C@H](O)CC1)CCCC2)c1cccnc1]

N-(3-(((1r,4r)-4-hydroxycyclohexyl) carbamoyl)-4,5,6,7-tetrahydrobenzo[b]thiophen-2-yl) nicotinamide

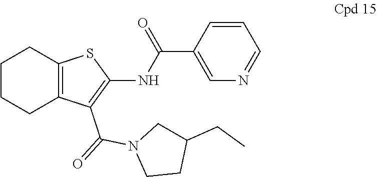 [SMILES: CCC1CCN(C(=O)c2c(NC(=O)c3cccnc3)sc3c2CCCC3)C1]

N-(3-(3-ethylpyrrolidine-1-carbonyl)-4,5,6,7-tetrahydrobenzo[b]thiophen-2-yl) nicotinamide

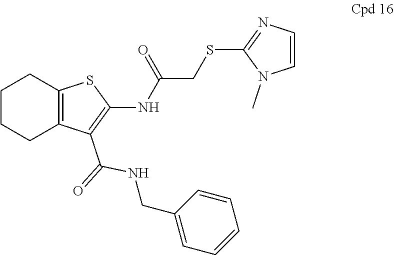 [SMILES: Cn1ccnc1SCC(=O)Nc1sc2c(c1C(=O)NCc1ccccc1)CCCC2]

N-benzyl-2-(2-((1-methyl-1H-imidazol-2-yl)thio) acetamido)-4,5,6,7-tetrahydrobenzo[b]thiophene-3-carboxamide

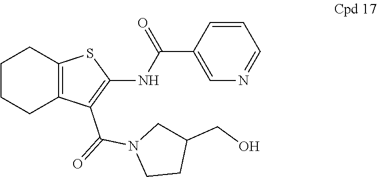 [SMILES: O=C(Nc1sc2c(c1C(=O)N1CCC(CO)C1)CCCC2)c1cccnc1]

N-(3-(3-(hydroxymethyl) pyrrolidine-1-carbonyl)-4,5,6,7-tetrahydrobenzo[b]thiophen-2-yl) nicotinamide

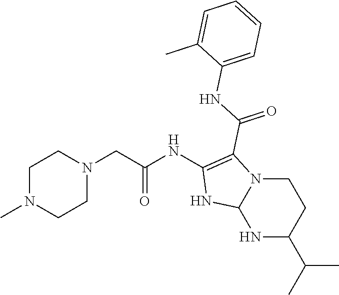 [SMILES: Cc1ccccc1NC(=O)C1=C(NC(=O)CN2CCN(C)CC2)NC2NC(C(C)C)CCN12]

7-isopropyl-2-(2-(4-methylpiperazin-1-yl) acetamido)-N-(o-tolyl)-1,5,6,7,8,8a-hexahydroimidazo[1,2-a]pyrimidine-3-carboxamide

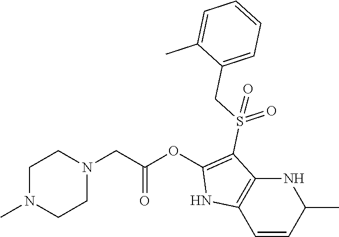

5-methyl-3-((2-methylbenzyl) sulfonyl)-4,5-dihydro-1H-pyrrolo[3,2-b]pyridin-2-yl 2-(4-methylpiperazin-1-yl)acetate

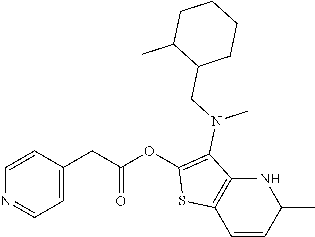 [SMILES: CC1C=Cc2sc(OC(=O)Cc3ccncc3)c(N(C)CC3CCCCC3C)c2N1]

5-methyl-3-(methyl((2-methylcyclohexyl)methyl)amino)-4,5-dihydrothieno[3,2-b]pyridin-2-yl 2-(pyridin-4-yl)acetate

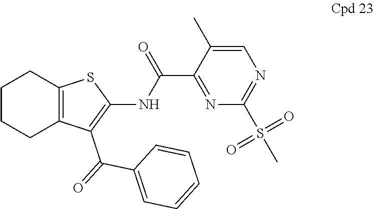 [SMILES: Cc1cnc(S(C)(=O)=O)nc1C(=O)Nc1sc2c(c1C(=O)c1ccccc1)CCCC2]

N-(3-benzoyl-4,5,6,7-tetrahydrobenzo[b]thiophen-2-yl)-5-methyl-2-(methylsulfonyl)pyrimidine-4-carboxamide

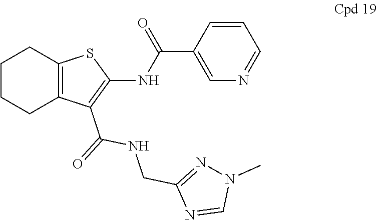 [SMILES: Cn1cnc(CNC(=O)c2c(NC(=O)c3cccnc3)sc3c2CCCC3)n1]

N-(3-(((1-methyl-1H-1,2,4-triazol-3-yl)methyl) carbamoyl)-4,5,6,7-tetrahydrobenzo[b]thiophen-2-yl) nicotinamide

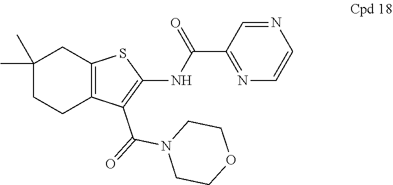 [SMILES: CC1(C)CCc2c(sc(NC(=O)c3cnccn3)c2C(=O)N2CCOCC2)C1]

N-(6,6-dimethyl-3-(morpholine-4-carbonyl)-4,5,6,7-tetrahydrobenzo[b]thiophen-2-yl) pyrazine-2-carboxamide

Another preferred inverse agonist is selected from the group consisting of:
N-(3-(4-benzylpiperazine-1-carbonyl)-4,5,6,7-tetrahydro-benzo[b]thiophen-2-yl)-2-fluorobenzamide;
N-(3-(benzylcarbamoyl)-4,5,6,7-tetrahydrobenzo[b]thiophen-2-yl)-5-chloro-2-(methylthio)pyrimidine-4-carboxamide;
5-chloro-N-(3-{[(1,1-dioxidotetrahydro-3-thienyl)amino]-carbonyl}-6-methyl-4,5,6,7-tetrahydro-1-benzothien-2-yl)-2-(methylthio)-4-pyrimidine-carboxamide;
N-(6-ethyl-3-{[(2-methylphenyl)amino]carbonyl}-4,5,6,7-tetrahydro-1-benzothien-2-yl)-1-methyl-1H-pyrazole-5-carboxamide;
N-{6-tert-butyl-3-[(4-methyl-1-piperazinyl) carbonyl]-4,5,6,7-tetrahydro-1-benzothien-2-yl}-2-fluorobenzamide;
2-fluoro-N-{6-methyl-3-[(4-methyl-1-piperazinyl) carbonyl]-4,5,6,7-tetrahydro-1-benzothien-2-yl}benzamide;
N-benzyl-2-[(trifluoroacetyl)amino]-4,5,6,7-tetrahydro-1-benzothiophene-3-carboxamide;
N-benzyl-2-({[(4-methyl-2-pyrimidinyl)thio]acetyl}amino)-4,5,6,7-tetrahydro-1-benzothiophene-3-carboxamide;
N-benzyl-2-[(1-piperidinylacetyl)amino]-4,5,6,7-tetrahydro-1-benzothiophene-3-carboxamide;
N-benzyl-2-{[(4-methyl-1-piperazinyl) acetyl]amino}-4,5,6,7-tetrahydro-1-benzothiophene-3-carboxamide;
N-benzyl-2-{[(4-methyl-1-piperidinyl) acetyl]amino}-4,5,6,7-tetrahydro-1-benzothiophene-3-carboxamide;
N-(3-(2-methylpyrrolidine-1-carbonyl)-4,5,6,7-tetrahydro-benzo[b]thiophen-2-yl) nicotinamide;
N-(3-((2R,4R)-2,4-dimethylpiperidine-1-carbonyl)-4,5,6,7-tetrahydrobenzo[b]thiophen-2-yl) nicotinamide;
N-(3-(((1r,4r)-4-hydroxycyclohexyl) carbamoyl)-4,5,6,7-tetrahydrobenzo[b]thiophen-2-yl) nicotinamide;
N-(3-(3-ethylpyrrolidine-1-carbonyl)-4,5,6,7-tetrahydro-benzo[b]thiophen-2-yl) nicotinamide;
N-benzyl-2-(2-((1-methyl-1H-imidazol-2-yl)thio) acetamido)-4,5,6,7-tetrahydrobenzo[b]thiophene-3-carboxamide;
N-(3-(3-(hydroxymethyl) pyrrolidine-1-carbonyl)-4,5,6,7-tetrahydrobenzo[b]thiophen-2-yl) nicotinamide;
N-(6,6-dimethyl-3-(morpholine-4-carbonyl)-4,5,6,7-tetrahydrobenzo[b]thiophen-2-yl) pyrazine-2-carboxamide;
N-(3-(((1-methyl-1H-1,2,4-triazol-3-yl)methyl) carbamoyl)-4,5,6,7-tetrahydrobenzo[b]thiophen-2-yl) nicotinamide;
(S)—N-(3-((1-cyanoethyl) carbamoyl)-4,5,6,7-tetrahydro-benzo[b]thiophen-2-yl) nicotinamide; and
N-(3-((piperidin-4-ylmethyl) carbamoyl)-4,5,6,7-tetrahydro-benzo[b]thiophen-2-yl) nicotinamide; N-(3-benzoyl-4, 5, 6, 7-tetrahydrobenzo[b]thiophen-2-yl) pyrazine-2-carboxamide; and
N-(3-benzoyl-4, 5, 6, 7-tetrahydrobenzo[b]thiophen-2-yl)-5-methyl-2 (methylsulfonyl)pyrimidine-4-carboxamide.
Another preferred inverse agonist is selected from the group consisting of:
N-(4-ethylphenyl)-3-(hydroxymethyl)-N-isobutyl-4-((tetrahydro-2H-pyran-4-yl) methoxy)benzenesulfonamide;
(7S)-N-{[5-(Ethylsulfonyl)-2-pyridinyl]methyl}-7-isopropyl-6-{[trans-4-(trifluoromethyl)cyclohexyl]methyl}-6,7-dihydro-5H-pyrrolo[3,4-b]pyridine-3-carboxamide;
N-{3-[(3-methylbut-2-en-1-yl) {methyl[trans-4-(pyridin-4-yl)cyclohexyl]carbamoyl}amino]phenyl}benzamide;
2-(1-(2,4-dichloro-3-((7-chloro-5-(trifluoromethyl)-1H-indol-1-yl)methyl)benzoyl) piperidin-4-yl) acetic acid;
(S)-2-(4-cyclopropyl-6-methylpyrimidin-5-yl)-8-(1-cyclopropylethyl)-6-(((5-(methylsulfonyl)pyridin-2-yl)methyl)amino) pteridin-7 (8H)-one;
(S)-6-(2,6-dimethylpyrimidin-4-yl)-N-(4-(ethylsulfonyl)benzyl)-6-methyl-5-oxo-5,6,7,8-tetrahydroquinoline-2-carboxamide;
2-(2-((S)-(3,5-dimethylisoxazol-4-yl) (hydroxy)methyl)benzofuran-5-yl)-N-((S)-(2,4-dimethylphenyl) (phenyl)methyl) acetamide;
N-(4-(1,1,1,3,3,3-hexafluoro-2-hydroxypropan-2-yl)phenyl)-N-(2,2,2-trifluoroethyl)benzenesulfonamide;
1,1,1,3,3,3-hexafluoro-2-(2-fluoro-4′-((4-(pyridin-4-ylmethyl) piperazin-1-yl)methyl)-[1,1′-biphenyl]-4-yl) propan-2-ol;
(1R,3aS,5aR,5bR,7aR,9S, 11aR, 11bR,13aR,13bR)-N-(3-(dimethylamino) propyl)-9-hydroxy-5a,5b,8,8,11a-pentamethyl-1-(prop-1-en-2-yl) icosahydro-3aH-cyclopenta[a]chrysene-3a-carboxamide; and

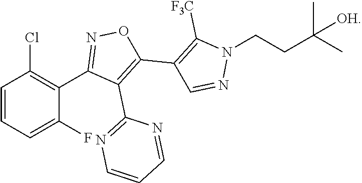 [SMILES: CC(C)(O)CCn1ncc(-c2onc(-c3c(F)cccc3Cl)c2-c2ncccn2)c1C(F)(F)F]

The use, further comprising one or more additional compounds selected from the group consisting of:
(a) a cytotoxic agent;
(b) an antimetabolite;
(c) an alkylating agent;
(d) an anthracycline;
(e) an antibiotic;
(f) an anti-mitotic agent;
(g) an hormone therapy;
(h) a signal transduction inhibitor;
(i) a gene expression modulator;
(j) an apoptosis inducer;
(k) an angiogenesis inhibitor
(l) an immunotherapy agent
(m) an Immune Checkpoint Inhibitor

and

a pharmaceutically acceptable carrier.

The cytotoxic agent is taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, Gemcitabine, Paclitaxel, FOLFIRINOX is a chemotheraphy combination to treat pancreatic cancer and is composed of leucovorin, folinic acid, flurouracil, irinotecan hydrochloride, oxaliplatin, analogs or homologs thereof, or a combination thereof.

The antimetabolites is methotrexate, 6-mercaptopurine, 6-thioguanine, cytarabine, 5-fluorouracil decarbazine, or a combination thereof.

The alkylating agent is mechlorethamine, thioepa chlorambucil, melphalan, carmustine (BSNU), lomustine (CCNU), cyclothosphamide, busulfan, dibromomannitol, streptozotocin, mitomycin C, cis-dichlorodiamine platinum (II) (DDP) cisplatin, or a combination thereof.

The anthracycline is daunorubicin, doxorubicin, or a combination thereof.

The antibiotic is dactinomycin, bleomycin, mithramycin, anthramycin (AMC), or a combination thereof.

The anti-mitotic agent is vincristine, vinblastine, or a combination thereof.

The signal transduction inhibitor is imatinib, trastuzumab, or a combination thereof.

The gene expression modulator is a siRNA, a shRNA, an antisense oligonucleotide, an HDAC inhibitor, or a combination thereof.

The immunotherapy agent is an Immune Checkpoint Inhibitors, a monoclonal antibody, a chimeric antigen receptors (CARs)-T-Cell, or a combination thereof.

The hormone therapy is an luteinizing hormone-releasing hormone (LHRH) antagonist.

The apoptosis inducers is a recombinant human TNF-related apoptosis-inducing ligand (TRAIL).

The angiogenesis inhibitors is sorafenib, sunitinib, pazopanib, everolimus or a combination thereof.

According to an embodiment, there is provided a whole blood diagnostic test to monitor cancer patients response to treatment and help predict patient outcomes; which comprises measuring total neutrophils, total lymphocytes and ratio of total neutrophils versus total lymphocytes in a blood sample before and after treatment with a ROR inverse agonist according to the present use; wherein increased in the ratio is indicative of a poor response to the treatment.

According to an embodiment, there is provided a whole blood diagnostic test to monitor cancer patients response to treatment and help predict patient outcomes; which comprises measuring RORC+ neutrophils, total lymphocytes and ratio of ROC+ neutrophils versus total lymphocytes in a blood sample before and after treatment with a ROR inverse agonist according to the present use; wherein increased in the ratio is indicative of a poor response to the treatment.

According to an embodiment, there is provided a whole blood diagnostic test to monitor cancer patients response to treatment and help predict patient outcomes; which comprises measuring RORC+ neutrophils and FOXP3+ Treg lymphocytes and ratio of RORC+ neutrophils versus FOXP3+ Treg lymphocytes in a blood sample before and after treatment with a ROR inverse agonist according to the present use; wherein increased in the ratio is indicative of a poor response to the treatment.

According to an embodiment, there is provided a whole blood diagnostic test to monitor cancer patients response to treatment and help predict patient outcomes; which comprises measuring RORC+ neutrophils and CD8+ Treg lymphocytes and ratio of RORC+ neutrophils versus CD8+ Treg lymphocytes in a blood sample before and after treatment with a ROR inverse agonist according to the present use; wherein increased in the ratio is indicative of a poor response to the treatment.

According to an embodiment, there is provided a whole blood diagnostic test to monitor cancer patients response to treatment and help predict patient outcomes; which comprises measuring ratio of RORC+ neutrophils versus FOXP3+ Treg lymphocytes, and ratio of RORC+ neutrophils versus CD8+ Treg lymphocytes in a blood sample before and after treatment with a ROR inverse agonist according to the present use; wherein increased in the ratio is indicative of a poor response to the treatment.

According to an embodiment, there is provided whole blood diagnostic test to monitor cancer patients response to treatment and help predict patient outcomes; which comprises measuring ratio of RORC+ neutrophils, to ratio of FOXP3+ Treg lymphocytes to CD8+ Treg lymphocytes in a blood sample before and after treatment with a ROR inverse agonist according to the present use; wherein increased in the ratio is indicative of a poor response to the treatment.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIGS. 1A-1B illustrate novel compounds of the present invention.

FIG. 2 illustrates compound 2 of the present invention.

FIG. 3 illustrates compound 10 of the present invention.

FIG. 4 illustrates a bar graph represents the % of IL17A+ cells also known as Th17 cells out of CD4+ cells known as Helper T cells. The peripheral blood mononuclear cells from 9 individuals were polarized (in-vitro) using CD3/CD28 dynabeads in presence of TGFβ, IL1B, IL6 and IL23 for 14 days. The cells from each individual was divided into two equal portions and one portion was treated with the Cpd10 at dose of 750 nM and the second portion was treated with the vehicle for 48 hours followed by PMA/ionomycin in presence of transport blocker for 6 hours. Cpd10 decreased the % of IL17A+CD4+ in treated compared to vehicle treated controls and measured by flow cytometry. Paired t-test, significant, P<0.05.

FIG. 5 illustrates a bar graph represents the % of IL21+ cells which includes all types of CD4+ cells secreting IL21 (e.g. Th17, Tfh). The peripheral blood mononuclear cells from 9 individuals were polarized (in-vitro) using CD3/CD28 dynabeads in presence of TGFβ, IL1β, IL6 and IL23 for 14 days. The cells from each individual was divided into two equal portions and one portion was treated with the Cpd10 at dose of 750 nM and the second portion was treated with the vehicle for 48 hours followed by PMA/ionomycin in presence of transport blocker for 6 hours. Cpd10 decreased the % of IL21+CD4+ in treated compared to vehicle treated controls measured by flow cytometry. Paired t-test Significant P<0.05.

FIG. 6 illustrates a bar graph represents the % of IL22+ cells which includes all types of CD4+ cells secreting IL22 (e.g. Th17, Th22). The peripheral blood mononuclear cells from 9 individuals were polarized (in-vitro) using CD3/CD28 dynabeads in presence of TGFβ, IL1β, IL6 and IL23 for 14 days. The cells from each individual was divided into two equal portions and one portion was treated with the Cpd10 at dose of 750 nM and the second portion was treated with the vehicle for 48 hours followed by PMA/ionomycin in presence of transport blocker for 6 hours. Cpd10 decreased the % of IL22+CD4+ in treated compared to vehicle treated controls measured by flow cytometry. Paired t-test, significant, P<0.05.

FIG. 7 illustrates a bar graph represents the % RORγt+ cells which includes all types of CD4+ cells expressing RORγt (e.g. Th17). The peripheral blood mononuclear cells from 9 individuals were polarized (in-vitro) using CD3/CD28 dynabeads in presence of TGFβ, IL1β, IL6 and IL23 for 14 days. The cells from each individual was divided into two equal portions and one portion was treated with the Cpd10 at dose of 750 nM and the second portion was treated with the vehicle for 48 hours followed by PMA/ionomycin in presence of transport blocker for 6 hours. Cpd10 increased the % of RORγt+ CD4+ in treated compared to vehicle treated controls measured by flow cytometry. Paired t-test, significant, P<0.05.

FIG. 8 illustrates the effect of different concentrations of Cpd 2 and Cpd 10 on the percentage of Th17 cells.* P<0.05 compared to response at zero concentration.

FIGS. 9 to 14 illustrate the synthesis of compounds of the present invention.

FIG. 15 illustrates RORγt co-activator recruitment was <10 nM for all the compounds tested (FIG. 17A). Th17 polarization in cell preparations was effectively inhibited with IC50's in the nM range (FIG. 17B). Several of our novel compounds (grey bars) compared favourably with GSK (black bar) (FIG. 17C). Epidermal hyperplasia, loss of skin appendages (hair follicles, sebaceous glands) and extensive dermal inflammation were present in DMSO controls and absent in treated recipients (1 mg/kg) (FIG. 17D).

FIG. 16 illustrates RORC expression in pancreatic cancer cell lines.

FIG. 17 illustrates the effect of compound 10 on cell viability. CellTiter-Glo: A luminescent cell viability assay based on quantifying ATP levels as a measurement of viable cells.

FIG. 18 illustrates the effect of compound 10 on Stem cell specific markers.

FIG. 19 illustrates the effect of compound 10 on Immune Checkpoint PDL1.

FIG. 20 illustrates the effect of compound 10 on Colony assay-HPAF II and on Colony assay-Miapaca.

FIG. 21A illustrates the effect of compound 10 on Aldefluor assay to measure ALDH activity in HPAF-II and in Miapaca-II (pancreatic cancer).

FIG. 21B illustrates the effects of RORC knock down on checkpoint “PDL1” expression in triple negative breast cancer cells.

FIG. 22 illustrates the effect of compound 10 on tumor volume in pancreatic cancer mouse model.

FIGS. 23A and B illustrate RORC expression in breast cancer cell lines using QPCR, Western Bolt and Flow cytometry.

FIG. 24 illustrates effect of increasing concentrations of compound 10 on cell viability. CellTiter-Glo: A luminescent cell viability assay based on quantifying ATP levels as a measurement of viable cells.

FIG. 25 illustrates the effect of 10 and 20 micromolar of compound 10 on Colony assay-MDA and on Colony assay-SUM.

FIG. 26 illustrates the effect of 25 mg/kg daily injections of compound 10 on tumour volume in a NSG mouse model. The tumours are allowed to grow to about a volume between 150-200 mm³ and then compound given daily for 2 weeks.

FIG. 27 illustrates the relative silencing of RORC on cell viability, cytotoxicity, and Caspase 3/7 a marker of Apoptosis.

FIG. 28 illustrates the effect of 5 micromolar on Cell colony forming units. Compound 10 significantly reduces the ability to form Colony forming units.

FIG. 29 illustrates in vitro immunofluorescence expression of RORy (A) and IL17 (B). HCC cell lines HuH-7 and Hepa1-6 express nuclear receptor RORy. Human HCC cell line HuH-7 and mouse HCC cell line Hepa1-6 were stained for RORy and IL-17 and expression was visualized via immunofluorescence microscopy. A) RORγ expression in HuH-7. RORγ in red and nucleus of cells in blue. B) IL-17 expression in HuH-7. IL-17 in red and nucleus of cells in blue. C) RORγ expression in Hepa1-6. RORγ in red and nucleus of cells in blue. D) IL-17 expression in Hepa1-6. IL-17 in red and nucleus of cells in blue.

FIG. 30 illustrates in vitro cell viability assay both human HuH-7 and mouse Hepa1-6 cell lines with increasing concentrations of compound 10. In the human cell line you see an effect with as low as 1mMol concentration. HuH-7 and Hepa1-6 cell viability assays. A) HuH-7 MTT assay reveals a significant and dose dependent difference in cell viability when subjected to varying concentrations of RORγ inhibitor C#10. B) Hepa1-6 MTT assay reveals a significant and dose dependent difference in cell viability when subjected to varying concentrations of RORγ inhibitor C#10. C) HuH-7 GFP viability assay reveals a significant and dose dependent difference in cell viability when subjected to varying concentrations of RORγ inhibitor C#10. D) Hepa1-6 GFP viability assay reveals a significant and dose dependent difference in cell viability when subjected to varying concentrations of RORγ inhibitor C#10.

FIG. 31 illustrates in vitro cell viability using another assay GFP (part of FIG. 30 in a bar graph).

FIG. 32 illustrates another in vitro viability assay Colony Formation Assay. We show that both cell lines are suppressed at 2.5 mMol concentration. HuH-7 and Hepa1-6 colony formation assays. A) HuH-7 colony formation assay reveals a significant and dose dependent difference in colony growth ability when subjected to varying concentrations of RORγ inhibitor C#10. B) Hepa1-6 colony formation assay reveals a significant and dose dependent difference in colony growth ability when subjected to varying concentrations of RORy inhibitor C#10.

FIG. 33 illustrates the effect of the compound in an in vivo mouse model of HCC injected in the flank. In many of the treated mice (over 3 weeks) no cancer is seen in most of the treated mice. HCC mouse model. Hepa1-6 cells (5×10⁶) were suspended in 100 μl of PBS and injected into the left flank of C57BL6 mice subcutaneously with 1 ml syringe. Mice were treated with a daily dose of RORγ inhibitor for 14 days. Subsequently, tumours were resected and tumour volume was measured. A) A decrease in tumour volume is visible over the course of 21 days in treated groups of mice compared to the control group. B) Total number of tumours at endpoint of experiment in control and treated groups. C) Representative pictures of tumours in control and treated groups at endpoint.

FIG. 34 illustrates Western blot analysis reveals RORγ expression in HuH-7 and Hepa1-6 cell lines. Western blot showing expression of RORγ (~55 kDa) in HuH-7 and Hepa1-6 cell lines. Mouse Thymus was used as a positive control. Actin (~42 kDa), was used as a control for protein loading and fraction purity.

FIG. 35 illustrates how treatment reduces the Neutrophil to Lymphocytes ratio in peripheral blood of mice (this is a pro-cancer finding and associated in cancer patients with poor outcome). this happens very early in this mouse cancer model.

FIG. 36 illustrates that Th17/Treg ratio increasing with the drug.

FIG. 37 illustrates that RORy inhibitor slows down (almost arrests) gastric cancer growth in mice (some mice the cancer stops growing).

FIG. 38 illustrates RORy inhibits intratumoral infiltration of RORγ+ Neutrophils.

FIGS. 39 and 40 illustrate that the RORy inhibitor induces a less Immunosuppressive environment by reducing FOXP3+ CD4 cells and increasing the CD8/Treg ratio.

FIG. 41 illustrates similar results with a lower dose of the drug (5 mg/kg).

FIG. 42 illustrates that this mouse gastric cancer has a very low RORy expression. This favours the conclusion that our RORy inhibitor acts on the cancer through a reduction of an Immunosuppressive microenvironment and conversion into a cytotoxic immune environment.

FIGS. 43 and 44 illustrate the Circulating RORγ+ Neutrophils are increased in the patients with increased Neutrophil/Lymphocyte ratio.

FIG. 45 illustrates that in the GE cancers there is increased RORγ+ neutrophils particularly in the advancing cancer periphery.

FIG. 46 illustrates that there is a lower patient survival in those that have a higher RORγ+ neutrophils in the cancer but the statistical power is not yet achieved due to small numbers (n=36 patients only).

FIG. 47 illustrates that 1117⁺ Neutrophils also are present in the cancer (il17+ is a surrogate marker of RORγ+

FIG. 48 illustrates that RORγ+ neutrophils express both II17 and PD-L1.

FIG. 49 illustrates that RORγ+ neutrophils possess pro-tumour properties listed in the slide especially II1A-tumour proliferation, HIF1A and PD-L1 (Cd274) Immunosuppression and PTEN-NETs formation.

FIG. 50 illustrates Balb/c skin graft survival in sensitized C57BL/6 recipients. Cpd 13 increase survival from 1 up to 3 weeks.

FIGS. 51-55 illustrate pathway enrichment analysis GSEA (Hallmark).

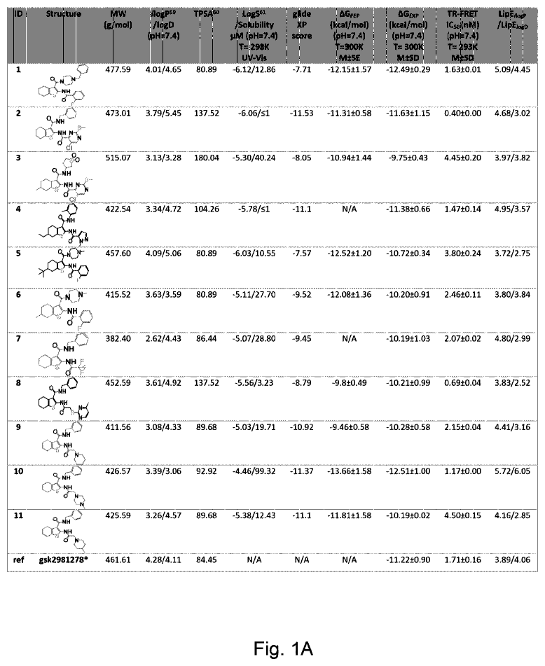

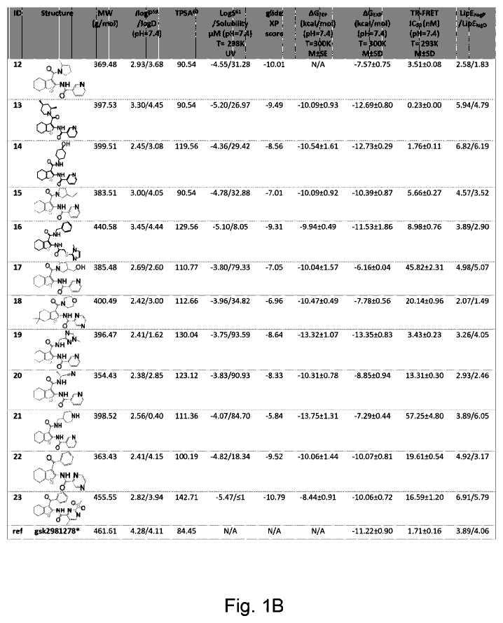

### DETAILED DESCRIPTION

The present invention relates to the use of a ROR inverse agonist or a pharmaceutically acceptable salt thereof for the manufacture of a medicament for modulating RORs and/or controlling autoimmune diseases and antibody mediated rejection in a patient.

According to an embodiment, there is provided the A method of preventing or treating a RORs mediated disease, autoimmune disease or antibody mediated rejection in a patient in a patient in need thereof, which comprises administering to said patient a therapeutically effective amount of a ROR inverse agonist, its pharmaceutically acceptable salt or stereoisomers thereof.

The RORs mediated disease or autoimmune disease is HIV, cancer, celiac disease, diabetes mellitus type 1, Graves' disease, inflammatory bowel disease, multiple sclerosis, psoriasis, rheumatoid arthritis, systemic lupus erythematosus, asthma, dermatitis, fatty liver disease, Crohn's disease, cardiovascular disease, inflammatory diseases, neurological disorder, multiple sclerosis, acute respiratory distress syndrome and arteriosclerosis.

The cancer is prostate cancer, breast cancer, ovarian cancer, multiple myeloma, brain cancer, glioma, lung cancer, salivary cancer, stomach cancer, thymic epithelial cancer, thyroid cancer, leukemia, melanoma, lymphoma, gastric cancer, pancreatic cancer, kidney cancer, bladder cancer, colon cancer, gastroesophageal adenocarcinoma and liver cancer.

The inverse agonist of RORs is a compound of Formula (I), (II), (III), or (IV), a pharmaceutically acceptable salts thereof, or stereoisomers thereof:

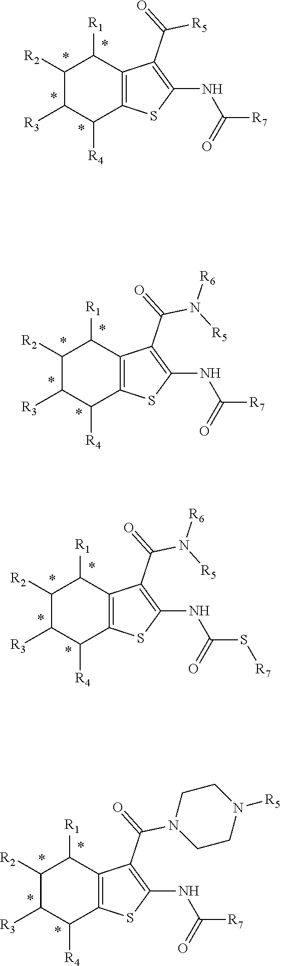

wherein,
R1, R2, R3, and/or R4 group(s) is/are H, halogen, NO2, 1-6 alkoxy, OH, NH2, 1-6 alkyl, 1-6 alkenyl, 1-6 haloalkyl, N-dialkyl, haloalkoxy, 1-6 hydroxyalkyl, and/or —CO2 (1-6 alkyl);
R5 is a substituted or unsubstituted five or six membered saturated or unsaturated heterocycle, aryl, alkylarene, halo aryl, ring substituted alkylarene, ring substituted alkylhexane, ring substituted alkylcyclopentane, haloaryl, benzene, phenyl, benzyl, pyridine, pyrimidine, pyridine, imidazole, diazole, triazole, thiadiazole, imidazolidine, thizolidine, pyrrolidine, piperazine, piperidine, pyridazine, pyrazine, triazine, 1H pyrrole, 2H pyrrole, pyrroline, pyrazolidine, pyrazoline, thiazole, isothiazole, isoxazole, haloalkyl, cyanoalkyl, methylpyrimidine, toluene, methylpyridine, methylimidazole, methyldiazole, methyltriazole, methylthiadiazole, methylimidazolidine, methylthizolidine, methylpyrrolidine, methylpiperazine, methylpiperidine, methylpyridazine, methylpyrazine, methyltriazine, methylpyrrole, methylpyrroline, methylpyrazolidine, methylpyrazoline, methylthiazole, methylisothiazole, methylisoxazole, or arylalkyl.
R6 is H, 1-6 alkyl, or may form a five or six ring structure with R5; and
R7 is a substituted or unsubstituted five or six membered saturated or unsaturated heterocycle, ring substituted alkylarene, ring substituted alkylhexane, ring substituted alkylcyclopentane, substituted haloaryl, substituted benzene, substituted phenyl, benzyl, pyrimidine, pyridine, imidazole, diazole, triazole, thiadiazole, imidazolidine, thizolidine, pyrrolidine, piperazine, aryl, halo aryl, alkylarene, piperidine, pyridazine, pyrazine, triazine, 1H pyrrole, 2H pyrrole, pyrroline, pyrazolidine, pyrazoline, thiazole, isothiazole, isoxazole, cyanoalkyl, methylpyrimidine, toluene, methylpyridine, methylimidazole, methyldiazole, methyltriazole, methylthiadiazole, methylimidazolidine, methylthizolidine, methylpyrrolidine, methylpiperazine, methylpiperidine, methylpyridazine, methylpyrazine, methyltriazine, methylpyrrole, methylpyrroline, methylpyrazolidine, methylpyrazoline, methylthiazole, methylisothiazole, methylisoxazole, or arylalkyl.

The invention includes the compounds as shown, and also includes (where possible) individual diastereomers, enantiomers, and epimers of the compounds, and mixtures of diastereomers and/or enantiomers thereof including racemic mixtures. Although the specific stereochemistries disclosed herein are preferred, other stereoisomers, including diastereomers, enantiomers, epimers, and mixtures of these may also be useful. Inactive or less active diastereoisomers and enantiomers are useful for scientific studies relating to the nuclear receptor targeting and the mechanism of activation.

According to another embodiment, the compounds of the present invention may be given directly to a patient in need of such treatment, using oral, intravenous, topical, intranasal, intrapulmonary, subcutaneous (slow release implant or patch), sublingual, inhalation or intramuscular administration.

ROR alpha and ROR gamma t are both expressed in lymphocytes during maturation. Irregularities in the expressions of ROR genes may lead to cancer, autoimmune disease. RORγt is the master regulator is Th17. Th17 cells produce many proinflammatory and pleiotropic cytokines IL17A, IL17F (can be also anti-inflammatory), IL21, IL22, IL24, IL26. In addition, Th17 cells secrete GM-CSF and TNF alpha. T cytotoxic 17 is also a type of lymphocyte that secrete all the aforementioned cytokines. T follicular helper cells secrete IL21 and are involved in many inflammatory diseases and types of cancers.

The invention presented herein, binds to ROR alpha and ROR gamma and block the production of IL17A, IL21, IL17F, IL24, IL26 and other cytokines produced by either T helper 17, T cytotoxic 17, T folicular helper cells, and those produced by all variants or cytotoxic T cells, and Helper T cells as well as Variants of B cells that produce the aforementioned cytokines.

Th17 cells have a role in macrophage and leukocytes recruitment to cause inflammation. RORC and RORA are highly expressed in certain types of cancer and in autoimmune diseases. Patients waitlisted on organ transplantation lists and who are categorized as Highly sensitized patients have a high activity of T helper 17, T folicular cells, and all T cell variants that produce the aforementioned cytokines. The invention would confer a therapeutic benefit to this category and to all other people who receive allogenic transplantation.

The current invention is useful in treatment of diseases involving the immune system whenever any of the aforementioned cytokines is involved either directly or indirectly in the activation of the immune system. The current invention is useful in treatment of cancers involving blood, Liver, breast, gastrointestinal tract.

Types of autoimmune diseases that may be treated by compounds of the present invention include, but are not limited to, cancer, celiac disease, diabetes mellitus type 1, Graves' disease, inflammatory bowel disease, multiple sclerosis, psoriasis, rheumatoid arthritis, systemic lupus erythematosus, asthma, dermatitis, fatty liver disease, Crohn's disease, cardiovascular disease, inflammatory diseases, neurological disorder, multiple sclerosis and arteriosclerosis.

Types of cancer that may be treated by compounds of the present invention include, but are not limited to, prostate cancer, breast cancer, brain cancer, glioma, lung cancer, salivary cancer, stomach cancer, thymic epithelial cancer, thyroid cancer, ovarian cancer, multiple myeloma, leukemia, melanoma, lymphoma, gastric cancer, kidney cancer, pancreatic cancer, bladder cancer, colon cancer and liver cancer.

### RORC Associated Diseases

Acromegaly, Rheumatoid Arthritis, Malignant neoplasm of breast, Candidiasis, Hypertrophic, Cardiomyopathy, Colorectal Carcinoma, Crohn Disease, Diabetes, Diabetes Mellitus, Non-Insulin-Dependent Diabetes Mellitus, Hamartoma, Inflammatory Bowel Diseases, Mycobacterium Infections, Systemic Scleroderma, Tuberous Sclerosis, Joint swelling, Lobomycosis, Hepatitis, Autoimmune Malignant neoplasm of lung, Growth Hormone-Secreting Pituitary Adenoma, Pre B-cell acute lymphoblastic leukemia, Malignant neoplasm of prostate, Prostate carcinoma, Breast Carcinoma, Carcinoma of lung, Familial multiple trichoepitheliomata, Primary malignant neoplasm of lung, Colorectal Cancer, FRIEDREICH ATAXIA 1, Allergic rhinitis (disorder), PACHYONYCHIA CONGENITA 3, Early Rheumatoid Arthritis, Necrotizing enterocolitis in fetus OR newborn, Autoimmune Diseases, Autoimmune Diseases, Roberts-SC phocomelia syndrome, Roberts-SC phocomelia syndrome, DYSFUNCTION-SKIN DISORDERS, DYSFUNCTION-SKIN DISORDERS, Obesity, Lymphedema, Tobacco Use Disorder, Inflammation, Sleep Disorders, Circadian Rhythm, IMMUNODEFICIENCY 42. Antibody mediated rejection, acute solid organ transplant rejection (liver, heart, lung, kidneys), chronic solid organ graft rejection (liver, heart, lung, kidneys), alloimmune hypersensitivity.

### RORA Associated Diseases

Bipolar Disorder, Mental Depression, Depressive disorder, Autistic Disorder, Stomach Neoplasms, Seasonal Affective Disorder, Anoxia, Age related macular degeneration, Atherosclerosis, Choriocarcinoma, Dyslipidemias, Tobacco Use Disorder, Choroidal Neovascularization, Sleep Disorders, Major Depressive Disorder, Carcinogenesis, Liver carcinoma, Bone Diseases, Malignant neoplasm of breast, Malignant tumor of colon, Colitis, Epithelial cyst, Hepatitis B, Hypothyroidism, Liver diseases, Liver neoplasms, Metabolic Diseases, Neuroblastoma, Degenerative polyarthritis, Osteoporosis, Dermatologic disorders, Post-Traumatic Stress Disorder, Liver Failure, Depressive Symptoms, Complete atrioventricular block, Hepatitis B, Chronic, Breast Carcinoma, Colon Carcinoma, Central neuroblastoma, Atrophy of cerebellum, Adenoviral infections, Autism Spectrum Disorders, Steatohepatitis, Acute-On-Chronic Liver Failure. Antibody mediated rejection, acute solid organ transplant rejection (liver, heart, lung, kidneys), chronic solid organ graft rejection (liver, heart, lung, kidneys), alloimmune hypersensitivity.

### IL17A Associated Diseases

Antibody mediated rejection, acute solid organ transplant rejection (liver, heart, lung, kidneys), chronic solid organ graft rejection (liver, heart, lung, kidneys), alloimmune hypersensitivity, Arthritis, Experimental, Autoimmune Diseases, Inflammation, Colitis, Pneumonia, Hypersensitivity, Graft-vs-Host Disease, Brain Ischemia, Chronic Alcoholic Intoxication, CNS disorder, Acute Promyelocytic Leukemia, Lymphoproliferative Disorders, Necrosis, Alcohol abuse, Chemical and Drug Induced Liver Injury, Chemical and Drug Induced Liver Injury, Periodontal Diseases, Chronic Obstructive Airway Disease, Spondylarthrosis, Myocardial Infarction, Diabetes Mellitus, Hyperalgesia, Myocardial Reperfusion Injury, Lipoid nephrosis, Pleurisy, Pulmonary Fibrosis, Staphylococcal Skin Infections, Acute Necrotizing Pancreatitis, Anti-Glomerular Basement Membrane Disease, Rheumatoid Arthritis, Asthma, Psoriasis, Arthritis, Systemic Lupus Erythematosus, Periodontitis, Multiple Sclerosis, Encephalomyelitis, Ulcerative Colitis, Degenerative polyarthritis, Inflammatory Bowel Diseases, Inflammatory Bowel Diseases, Behcet Syndrome, Malignant neoplasm of stomach, Gingivitis, Sjogren's Syndrome, Juvenile arthritis, Candidiasis, Chronic Mucocutaneous Uveitis, Helicobacter Infections, Crohn Disease, Atopic Dermatitis, Celiac Disease, Systemic Scleroderma, Dermatitis, Cutaneous T-cell Lymphoma, Malignant neoplasm of breast, Carcinogenesis, Stomach Carcinoma, Multiple Myeloma, Job Syndrome, Arteriosclerosis, Colorectal Carcinoma, Colorectal Cancer, Inflammatory dermatosis, Bronchiolitis Obliterans, Keratitis, Coronary Artery Disease, Bronchiolitis, Cystic Fibrosis, Rheumatoid Nodule, Respiratory Syncytial Virus Infections, Bone Diseases, Dyspepsia, Endometriosis, Alcoholic Hepatitis, Bone necrosis, Rhinitis, Thrombocytopenia purpura, Ovarian neoplasm, Infection, Psoriatic Arthritis, Viral Bronchiolitis, Stomach Neoplasms, Tobacco Use Disorder, Mycoses, Lupus Erythematosus, Eczema, Lupus Vulgaris, Lupus Erythematosus, Discoid Virus Diseases, Bone destruction, Capillary malformation (disorder), Liver carcinoma, Neutrophilia (disorder), Diabetes Mellitus Insulin-Dependent, Neoplasm Metastasis, Neoplasm Metastasis, Dermatologic disorders, Ankylosing spondylitis, Tuberculosis, Liver Fibrosis, Pneumonitis, Bacterial Infections, Non-Small Cell Lung Carcinoma, Eosinophilia, Lupus Nephritis, Osteopenia, Allergic asthma, Malignant neoplasm of lung, Cervix carcinoma, Superficial ulcer, Breast Carcinoma, Carcinoma of lung, Inflammatory disorder, Primary malignant neoplasm of lung, Eosinophilic disorder, DYSFUNCTION-SKIN DISORDERS, Skin Erosion, Candidiasis, Mucocutaneous candidiasis, Malignant tumor of colon, Malignant tumor of cervix, Histiocytosis Langerhans-Cell, Lung Neoplasms, Primary Sjogren's syndrome, Age related macular degeneration, Sepsis, Psoriasiform eczema, Chronic Periodontitis, Persistent embryonic structure, Helicobacter pylori (H. pylori) infection in conditions classified elsewhere and of unspecified site, Hepatitis B Chronic, Colon Carcinoma, Cervical cancer, Refractory anemias, Primary biliary cirrhosis, Dermatomyositis, Diabetes, Diabetes Mellitus, Encephalitis (St. Louis), Hepatitis B, Influenza, Chronic Lymphocytic Leukemia, Mucocutaneous Lymph Node Syndrome, Mycobacterium Infections, Nasal Polyps, Septicemia, Ocular Toxoplasmosis, Chagas Disease, Pulmonary Tuberculosis, Uveomeningoencephalitic Syndrome, Polyglandular Type I Autoimmune Syndrome, Complete Atrioventricular block, Tumor Progression, Chronic small plaque psoriasis, Hyperactive behavior, Hashimoto Disease, Infectious disease of lung, Acute Coronary Syndrome, Mammary Neoplasms, Tumor, Angiogenesis, Allergic rhinitis (disorder), Autoimmune arthritis, Adenoma, Alveolar Bone Loss, Extrinsic allergic alveolitis, Alzheimer's Disease, Bacterial Pneumonia, Dengue Fever, Fatty Liver, Gastritis, Glioblastoma, Glioma, Glomerulonephritis, Grave's Disease, Hamartoma Syndrome (Multiple), Hepatitis, Hepatitis A, Hypertensive disease, Arthropathy, Liver Cirrhosis, Mycosis Fungoides, Obesity, Ovarian Carcinoma, Pelvic Pain, Peritonitis, Prune Belly Syndrome, Cerebrovascular accident, Giant Cell Arteritis, Ulcer, Vitiligo, Corneal pannus, Acute myocardial infarction, Abdominal Aortic Aneurysm, Lyme Arthritis, Parasitemia, Psoriasis vulgaris, MRSA-Methicillin resistant Staphylococcus aureus infection, Respiratory syncytial virus (RSV) infection in conditions classified elsewhere and of unspecified site, Acute anterior uveitis, Neurological disability, Pelvic pain female, Acute GVH disease, Malignant neoplasm of ovary, Fungal keratitis, Chronic inflammatory disorder, Tumor Immunity, Painful Bladder Syndrome, VITILIGO-ASSOCIATED MULTIPLE AUTOIMMUNE DISEASE SUSCEPTIBILITY 1 (finding), Steatohepatitis, Ischemic Cerebrovascular Accident, Autoimmune polyendocrinopathy syndrome type 1, Interstitial lung fibrosis, Acne Vulgaris, Acquired Immunodeficiency Syndrome, Amyotrophic Lateral Sclerosis, Anemia, Aneurysm, Barrett Esophagus, Malignant neoplasm of urinary bladder, Bladder Neoplasm, Bronchiectasis, Brucellosis, Oral candidiasis, Malignant neoplasm of endometrium, Squamous cell carcinoma, Cardiovascular Diseases, Intracranial Aneurysm, Uterine Cervical Neoplasm, Colonic Diseases, Colonic Neoplasms, Common Variable Immunodeficiency, Allergic Conjunctivitis, Corneal Diseases, Coronary Arteriosclerosis, Coronary heart disease, Dejerine-Sottas Disease (disorder) Delirium, Diabetes Mellitus, Non-Insulin-Dependent, Diabetic Nephropathy, Enterovirus Infections, Epilepsy, Epithelial hyperplasia, Eye Infections, Giant Cell Tumors, Gonorrhea, Chronic granulomatous disease, Guillain-Barre Syndrome, Cardiac Arrest, Severe Dengue, Chronic Hepatitis, Hepatitis C, Herpes Simplex Infections, Herpesviridae Infections, HIV Infections, Hodgkin Disease, Hyperlipidemia, Hypothyroidism, Immune System Diseases, Kidney Diseases, Acute Kidney Failure, Chronic Kidney Failure, Leishmaniasis, Cutaneous Leishmaniasis, Infection by Leishmania braziliensis, Visceral Leishmaniasis, Leprosy, Lepromatous, Chronic Myeloid Leukemia, Liver diseases, Alcoholic Liver Diseases, Lyme Disease, Lymphoma, Malaria, Cerebral Malaria, Marinesco-Sjogren syndrome, Melanoma, Mitral Valve Stenosis, Myocarditis, Nervous system disorder, Nodule, Osteosarcoma, Pain, Pustulosis of Palms and Soles, Panuveitis, Periodontitis (Juvenile), Pituitary Adenoma, Pneumococcal Infections, Polyps, Prostatitis, Pulmonary Eosinophilia, Henoch-Schoenlein Purpura, Adult Respiratory Distress Syndrome, Retroviridae Infections, Salmonella infections, Sarcoidosis, Pulmonary Sarcoidosis, Schizophrenia, Sezary Syndrome, Situs Inversus, Gastric ulcer, Synovitis, Thrombocytopenia, Thymoma, Thyroid Diseases, Trachoma, Tuberous Sclerosis, Urticaria, Anterior uveitis, Intermediate Uveitis, Vaccinia, Vascular Diseases, Vasculitis, Intestinal Volvulus, Malignant Pleural Effusion, Adult-Onset Still Disease, Autoimmune Polyendocrinopathies, Liver Failure, Chronic sinusitis, Deep Vein Thrombosis, Chronic pain, Active tuberculosis, Secondary Sjogren's syndrome, Adenocarcinoma of lung (disorder), Lobomycosis, Pustular psoriasis, Systemic candidiasis, Acute myocarditis, Intervertebral disc disorder, Adrenoleukodystrophy, Allergic contact Dermatitis, Hypopigmentation disorder, Autoimmune thyroid disease, Autoimmune Diabetes, Interstitial Lung Diseases, Idiopathic Hypereosinophilic Syndrome, Ki-1+ Anaplastic Large Cell Lymphoma, Hemangioma of liver, Papillary thyroid carcinoma, Ankle arthritis, Autoimmune Hepatitis, Lewis lung Carcinoma, Chronic urticaria, Epithelial hyperplasia of skin, Childhood asthma, Congenital emphysema, Infantile Colic, Typhlocolitis, Acquired aplastic anemia, Viral myocarditis, Rhinovirus infection, Infection by Candida albicans, Gastric Adenocarcinoma, Tumor necrosis, Secondary bacterial pneumonia, Asthmatic pulmonary eosinophilia, Lofgrens syndrome, Stable angina, Anterior myocardial infarction, Dissection of aorta, Autoimmune enteropathy, Generalized pustular psoriasis, Malignant neoplasm of prostate, Hematologic Neoplasms, Leukocyte adhesion deficiency type 1, Non-alcoholic Fatty Liver Disease, Idiopathic crescentic glomerulonephritis, Primary antiphospholipid syndrome, Endometrial Carcinoma, Pricking of skin, Combined immunodeficiency, Central Sleep Apnea, Recurrent tumor, Neurodegenerative Disorders, X-Linked Lymphoproliferative Disorder, Osteosarcoma of bone, Aqueous Humor Disorders, Prostate carcinoma, Carcinoma of bladder, Airway disease, Acne, Dissecting aneurysm of the thoracic aorta, Viral respiratory infection, Corneal infection, Middle Cerebral Artery Occlusion, Acute pneumonia, Allergic symptom, Type I Cockayne Syndrome, Idiopathic Inflammatory Myopathies, Gluten sensitivity, Helicobacter pylori infection, Arteriopathic disease, Pseudomonas aeruginosa infection, Endothelial dysfunction, Chronic candidiasis, Plasmodium vivax infection, Mycosis fungoides/Sezary syndrome, Chronic graft-versus-host disease, Pancolitis, Vascular inflammations, Invasive Ductal Breast Carcinoma, Lymphocytic infiltration, Thyroid Gland Spindle Cell Tumor with Thymus-Like Differentiation, Granulocytosis, Pancreatic intraepithelial neoplasia, Sporadic Breast Carcinoma, Cardiac fibrosis, Familial Idiopathic Cardiomyopathy, Latent Tuberculosis, Cirrhosis, Minimal Change Nephrotic Syndrome, Non-Neoplastic Disorder, Severe Sepsis, DOSAGE-SENSITIVE SEX REVERSAL, CHRONIC MYELOPROLIFERATIVE DISORDER, CHRONIC MYELOPROLIFERATIVE DISORDER WITH EOSINOPHILIA, Childhood Ataxia with Central Nervous System Hypomyelinization, AMYOTROPHIC LATERAL SCLEROSIS 1, Sporadic Amyotrophic Lateral Sclerosis, Premature Coronary artery disease, Type I Crossed Polydactyly, Uterine Corpus Cancer, Extranodal NK-T-Cell Lymphoma, Photoreceptor degeneration, Chronic Lyme disease, Cryopyrin-Associated Periodic Syndromes, Chronic kidney disease stage 5, Destructive Arthritis, Adiponectin Deficiency, Sessile Serrated Adenoma/Polyp, Dianzani autoimmune lymphoproliferative syndrome, Autosomal dominant Hyper-Immunoglobulin E Syndrome, Immune reconstitution inflammatory syndrome[IRIS], Nonalcoholic Steatohepatitis, MYELODYSPLASTIC SYNDROME, Granulomatosis with polyangiitis, Familial Hypophosphatemic Rickets, Allergic disposition, Acute-On-Chronic Liver Failure, Aspirin exacerbated respiratory disease, Pre-renal acute kidney injury, Post-Treatment Lyme Disease Syndrome, Early Rheumatoid Arthritis, Membranous Lupus Nephritis, Necrotizing enterocolitis in fetus OR newborn.

### IL21 Associated Diseases

Celiac Disease, Systemic Lupus Erythematosus, COMMON VARIABLE 11 IMMUNODEFICIENCY, Experimental Autoimmune Encephalomyelitis, Periodontal Diseases, Rheumatoid Arthritis, Autoimmune Diseases, Insulin-Dependent Diabetes Mellitus, Asthma, Ulcerative Colitis, Crohn Disease, Infection, Multiple Sclerosis, Sezary Syndrome, Follicular Lymphoma, Acquired Immunodeficiency Syndrome, Hodgkin Disease, Falciparum Malaria, Juvenile arthritis, Chronic Lymphocytic Leukemia, Addison Disease, Alopecia Areata, Viral Bronchiolitis, Immediate hypersensitivity, Respiratory Syncytial Virus Infections, Inflammatory Bowel Diseases, Lupus Vulgaris, Discoid Lupus Erythematosus, Psoriasis, Lupus Erythematosus, Hepatitis B, Multiple Myeloma, Virus Diseases, Diffuse Large B-Cell Lymphoma, Immune thrombocytopenia purpura, Complete atrioventricular block, Persistent embryonic structure, Malignant neoplasm of breast, Malignant tumor of colon, Colitis, Common Variable Immunodeficiency, Atopic Dermatitis, Diabetes, Diabetes Mellitus, Eczema, Grave's Disease, Adult T-Cell Lymphoma/Leukemia, Lymphoma, Malignant neoplasm of stomach, Ovarian Carcinoma, Schistosomiasis, B-Cell Lymphomas, Primary Sjogren's syndrome, Chronic Hepatitis B, Chronic Hepatitis C, Breast Carcinoma, Colon Carcinoma, Stomach Carcinoma, Malignant neoplasm of ovary, X-Linked Combined Immunodeficiency Diseases, Pseudohyperkalemia Cardiff, Acquired Hypogammaglobulinemia, Aplastic Anemia, Arthritis, Burkitt Lymphoma, Cerebral Infarction, Echinococcosis, Glioblastoma, Glioma, Graft-vs-Host Disease, Hepatitis, HIV Infections, Angioimmunoblastic Lymphadenopathy, Immunologic Deficiency Syndromes, Liver diseases, Liver neoplasms, Chronic Obstructive Airway Disease, Melanoma, Mikulicz Disease, Nasal Polyps, Neoplasm Metastasis, Neuroblastoma, Degenerative polyarthritis, Polyps, Henoch-Schoenlein Purpura, Schizophrenia, Systemic Scleroderma, Sialadenitis, Cerebrovascular accident, Toxoplasmosis, Tuberous Sclerosis, Uveomeningoencephalitic Syndrome, Cutaneous T-cell Lymphoma, Severe Combined Immunodeficiency, Sicca Syndrome, Oral Ulcer, Idiopathic pulmonary hypertension, Dacryoadenitis, Allergic asthma, Malnutrition, Autoimmune thyroid disease, Brain cyst, Pancreatic carcinoma, Collagenous Colitis, Bone destruction, Autoimmune thrombocytopenia, Epithelial hyperplasia of skin, Familial lichen amyloidosis, Acquired aplastic anemia, Thrombocytopenia due to platelet alloimmunization, Disseminated neuroblastoma, Solid tumor, Malignant lymphoma (lymphocytic, intermediate differentiation, diffuse), Hematologic Neoplasms, Lymphocytic Colitis, Hydatids, Thymic alymphoplasia, Leukemogenesis, Hashimoto Disease, Central neuroblastoma, Allergic symptom, Acute Cerebrovascular Accidents, Pancolitis, Cerebral Ischemia, Ischemic stroke, Progressive multiple sclerosis, Stage 4S neuroblastoma, Inflammatory disorder, ALK negative Anaplastic large cell lymphoma, Sjogren's Syndrome, Benign Prostatic Hyperplasia, Liver carcinoma, Idiopathic pulmonary arterial hypertension, Ischemic Cerebrovascular Accident, Acute-On-Chronic Liver Failure, Early Rheumatoid Arthritis, Selective immunoglobulin A deficiency. Antibody mediated rejection, acute solid organ transplant rejection (liver, heart, lung, kidneys), chronic solid organ graft rejection (liver, heart, lung, kidneys), alloimmune hypersensitivity.

### IL22 Associated Diseases

Autoimmune Hepatitis, Middle Cerebral Artery Infractions, Chemical and Drug Induced Liver Injury, Asthma, Myocarditis, Crohn Disease, Psoriasis, Inflammatory Bowel Diseases, Hepatitis C Infection, Liver carcinoma, Autoimmune Diseases, Rheumatoid Arthritis, Systemic Lupus Erythematosus, HIV Infections, Chronic Mucocutaneous Candidiasis, Pneumonia, Inflammation, Colitis, Ulcerative Colitis, Viral Bronchiolitis, Colonic Neoplasms, Respiratory Syncytial Virus Infections, Atopic Dermatitis, Eczema, Chronic Lymphocytic Leukemia, Colon Carcinoma, Inflammatory dermatosis, Malignant tumor of colon, Dermatitis, Multiple Sclerosis, Carcinogenesis, Celiac Disease, Malignant neoplasm of stomach, Multiple Myeloma, Primary Sjogren's syndrome, Stomach Carcinoma, Amyloidosis, Arthritis, Colorectal Carcinoma, Diabetes Mellitus (Insulin-Dependent), Hepatitis A, Hepatitis B, Liver diseases, Liver neoplasms, Chronic Obstructive Airway Disease, Mycoses, Tuberculosis, Virus Diseases, Cutaneous T-Cell Lymphoma, Polyglandular Type I Autoimmune Syndrome, Complete atrioventricular block, Immune Thrombocytopenia purpura, Colorectal Cancer, DYSFUNCTION-SKIN DISORDERS, Bacterial Infections, Behcet Syndrome, Malignant neoplasm of breast, Candidiasis, Non-Small Cell Lung Carcinoma, Glioblastoma, Grave's Disease, Lymphoma, Ovarian Carcinoma, Schistosomiasis, Sezary Syndrome, Dermatologic disorders, Uveitis, Diffuse Large B-Cell Lymphoma, Malnutrition, Ki-1+ Anaplastic Large, Cell Lymphoma, Liver Fibrosis, Persistent embryonic structure, Hyperactive behavior, Breast Carcinoma, Helicobacter pylori infection, Malignant neoplasm of ovary, Sjogren's Syndrome, Acute colitis, Abscess, Adenocarcinoma, Adenovirus Infections, Alzheimer's Disease, Aortic Valve Insufficiency, Bacterial Pneumonia, Burkitt Lymphoma, Oral candidiasis, Candidiasis of vagina, Squamous cell carcinoma, Cerebral Infarction, Colonic Diseases, Cytomegalovirus Infections, Dejerine-Sottas Disease (disorder), Dengue Fever, Diabetes, Diabetes Mellitus, Non-Insulin-Dependent Diabetes Mellitus, Diarrhea, Echinococcosis, Erythema Nodosum, Gastroenteritis, Gingival Diseases, Glioma, Graft-vs-Host Disease, Severe Dengue, Angioimmunoblastic Lymphadenopathy, Immunologic Deficiency Syndromes, Influenza, Leukemia Myelocytic, Alcoholic Liver Diseases, Lupus Vulgarism Discoid Lupus Erythematosus, Lupus Nephritis, melanoma, Nasal Polyps, Nephritis, Neuroblastoma, Nodule, Obesity, Degenerative polyarthritis, Pustulosis of Palms and Soles, Pancreatitis, Periodontal Diseases, Polyps, Henoch-Schoenlein Purpura, Rotavirus Infections, Schizophrenia, Systemic Scleroderma, Septicemia, Skin lesion, Ankylosing spondylitis, Cerebrovascular accident, Thyroid Diseases, Toxoplasmosis, Tuberous Sclerosis, Urticaria Pigmentosa, Viral hepatitis, Vitiligo, B-Cell Lymphomas, Severe Combined Immunodeficiency, Sicca Syndrome, Retinal Vasculitis, Lobomycosis, Idiopathic pulmonary hypertension, Corneal pannus, Hypopigmentation disorder, Hidradenitis Suppurativa, Intestinal infectious disease (disorder), Autoimmune thyroid disease, Tumor Progression, brain cyst, Pancreatic carcinoma, Collagenous Colitis, Papillary thyroid carcinoma, Malignant neoplasm of lung Mucosa-Associated Lymphoid Tissue, Lymphoma, Sepsis, Psoriasiform eczema, Psoriasis vulgaris, Epithelial hyperplasia of skin, Typhlitis, Familial lichen amyloidosis, Acquired aplastic anemia, Disseminated neuroblastoma, Capillary malformation (disorder), Generalized pustular psoriasis, MRSA Methicillin resistant Staphylococcus aureus infection, Helicobacter pylori (H. pylori) infection in conditions classified elsewhere and of unspecified site, Hematologic Neoplasms, Microscopic Colitis, Lymphocytic Colitis, Proliferative nephritis unspecified, Chronic small plaque psoriasis, Lupus Erythematosus, Pricking of skin, Hydatids, Chronic Hepatitis B, Chronic Hepatitis C, Leukemogenesis, Hashimoto Disease, Carcinoma of lung, Central neuroblastoma, Acute Cerebrovascular Accidents, Acute GVH disease, Cerebral Ischemia, Intraabdominal Infections, Stage 4S neuroblastoma, X-Linked Combined Immunodeficiency Diseases, Inflammatory disorder, Chronic inflammatory disorder, Primary malignant neoplasm of lung, Anaplastic, ALK negative large cell lymphoma, ALK-Positive Anaplastic Large Cell Lymphoma, Gastric Mucosa-Associated Lymphoid Tissue Lymphoma, Intestinal Graft Versus Host Disease, Benign Prostatic Hyperplasia, Chromosome 11p11.2 Deletion Syndrome, IMMUNE SUPPRESSION, VITILIGO-ASSOCIATED MULTIPLE AUTOIMMUNE DISEASE SUSCEPTIBILITY 1 (finding), DOSAGE-SENSITIVE SEX REVERSAL, PEELING SKIN SYNDROME, Pseudohyperkalemia Cardiff, Immune reconstitution inflammatory syndrome[IRIS], Idiopathic pulmonary arterial hypertension, Enthesitis-Related Arthritis, Ulcerative colitis in remission, Pneumonitis, Early Rheumatoid Arthritis. Antibody mediated rejection, acute solid organ transplant rejection (liver, heart, lung, kidneys), chronic solid organ graft rejection (liver, heart, lung, kidneys), alloimmune hypersensitivity.

### IL24 Associated Disease

Lung Neoplasms, Pancreatic Neoplasm, Major Depressive Disorder, Mammary Neoplasms, Prostatic Neoplasms, Spontaneous abortion, Unipolar Depression, Periodontal Diseases, Melanoma, Liver carcinoma, Glioma, Carcinoma of lung, Malignant neoplasm of lung, Malignant neoplasm of breast, Prostate carcinoma, Breast Carcinoma, Primary malignant neoplasm of lung, Metastatic melanoma, Malignant neoplasm of prostate, Glioblastoma, Chronic Lymphocytic Leukemia, Squamous cell carcinoma, Adenocarcinoma, Rheumatoid Arthritis, Non-Small Cell Lung Carcinoma, Neoplasm Metastasis, Asthma, Viral Bronchiolitis, Hepatitis C, HIV Infections, Respiratory Syncytial Virus Infections, Ovarian Carcinoma, Pancreatic carcinoma, Malignant neoplasm of ovary, Colorectal Carcinoma, Malignant neoplasm of pancreas, Glioblastoma Multiforme, Colorectal Cancer, leukemia, Psoriasis, Epithelial ovarian cancer, Tumor Angiogenesis, Renal Cell Carcinoma, Liver and Intrahepatic Biliary Tract Carcinoma, solid tumor, Malignant neoplasm of liver, Malignant Glioma, Renal carcinoma, Autoimmune Diseases, Ulcerative Colitis, Crohn Disease, Inflammatory Bowel Diseases, Influenza, Acute lymphocytic, leukemia, Myeloid Leukemia, Retinoblastoma, Secondary malignant neoplasm of lung, Carcinogenesis, Stomach Carcinoma, ovarian neoplasm, skin disorders, Malignant neoplasm of urinary bladder, Bladder Neoplasm, Brain Neoplasms, Malignant tumor of colon, Malignant tumor of cervix, Dermatitis, Lymphoid leukemia, Acute Myelocytic Leukemia, Systemic Lupus Erythematosus, Malignant neoplasm of stomach, Mesothelioma, Experimental Neoplasms, Neuroblastoma, Pustulosis of Palms and Soles, Juvenile Periodontitis, Salmonella infections, Dermatologic disorders, Typhoid Fever, Urinary tract infection, Virus Diseases, Vitiligo, Tumor Progression, Acute urinary tract infection, Epithelial hyperplasia of skin, Gastric Adenocarcinoma, Conventional (Clear Cell) Renal Cell Carcinoma, Cervix carcinoma, Generalized pustular psoriasis, Malignant mesothelioma, Multiple malignancy, Disseminated Malignant Neoplasm, Neuropathy, Squamous cell carcinoma of skin, Secondary malignant neoplasm of lymph node, Colon Carcinoma, Carcinoma of bladder, Central neuroblastoma, MICROPHTHALMIA (SYNDROMIC 7), Malignant Pleural Mesothelioma, Adenoviral infections, B Lymphoblastic Leukemia/Lymphoma, Squamous cell carcinoma of the head and neck, Mesothelioma (malignant, clinical disorder) (disorder), Pancreatic Ductal Adenocarcinoma, Mammary Tumorigenesis, VITILIGO-ASSOCIATED MULTIPLE AUTOIMMUNE DISEASE SUSCEPTIBILITY 1 (finding), Precursor Cell, Lymphoblastic Leukemia Lymphoma, Mechanical Allodynia, Inflammatory dermatosis, MIXED LINEAGE LEUKEMIA, Persistent Oligoarticular Juvenile Idiopathic Arthritis, Cervical cancer.

### IL26 Associated Diseases

Rheumatoid Arthritis, Crohn Disease, Asthma, Viral Bronchiolitis, Ulcerative Colitis, Insulin-Dependent Diabetes Mellitus, Inflammatory Bowel Diseases, Multiple Sclerosis, Respiratory Syncytial Virus Infections, Tobacco Use Disorder, Chronic ulcerative colitis, Bronchiolitis Obliterans, Graft-vs-Host Disease, Malignant neoplasm of stomach, Pulmonary Fibrosis, Tuberculosis, Stomach Carcinoma, Chronic graft-versus-host disease.

### Abbreviations

Abbreviations and terms that are commonly used in the fields of organic chemistry, medicinal chemistry, pharmacology, and medicine and are well known to practitioners in these fields are used herein. Representative abbreviations and definitions are provided below:

Ac is acetyl[CH₃C(O)—], AC₂O is acetic anhydride; APC is antigen-presenting cell; 9-BBN is 9-borabicyclo[3.3.1]nonane; Bn is benzyl; BOC is tert Butyloxycarbonyl; DIAD is diisopropylazodicarboxylate; DIBAL is diisobutylaluminum hydride; DMF is N,N-dimethylformamide; DMSO is dimethyl sulfoxide; EDAC (or EDC) is 1-ethyl-3-[3-(dimethylamino) propyl]-carbodiimide HCl; Et₃N is triethylamine; Et is ethyl; EtOAc is ethyl acetate; EtOH is ethanol; 3-F-Ph is 3-fluorophenyl, HCl is hydrochloric acid; HOBt is 1-hydroxybenzotriazole; HPLC is high performance liquid chromatography; LCMS is HPLC with mass Spectral detection; LG is leaving group; M is molar; mmol is millimole; Me is methyl; MeOH is methanol; MsCl methanesulfonyl chloride; N is normal; NaHMDS is sodium hexamethyldisiliazide; NaOAc is sodium acetate; NaOtBu is sodium tert-butoxide; NMO is N-methylmorpholine N oxide; NMP is N Methyl pyrrolidinone; Pd(dba)₂ is tris(dibenzylideneacetone) dipalladium; PdCl₂ (Ph₃P)₂ is dichlorobis-(triphenylphosphene) palladium; PG Denotes an unspecified protecting group; Ph is phenyl; PhMe is toluene; PPh₃ is triphenylphosphine; PMB is para-methoxybenzyl; RT is room temperature; TBAF is tetrabutyl ammonium fluoride; TBS is tert-butyldimethylsilyl; tBu is tert-butyl; Tf is triflate; TFA is trifluoroacetic acid; THF is tetrahydrofuran; TLC is thin layer chromatography; TMS is trimethylsilyl; TPAP is tetrapropylammonium perruthenate.

### Definitions

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C3-10, the term alkyl also includes cycloalkyl groups, and combinations of linear or branched alkyl chains combined with cycloalkyl structures. When no number of carbon atoms is specified, C1-6 is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C1-6 alkoxy), or any number within this range[i.e., methoxy (MeO—), ethoxy, isopropoxy, etc.].

The term “alkylthio” refers to straight or branched chain alkylsulfides of the number of carbon atoms specified (e.g., C1-6 alkylthio), or any number within this range[i.e., methylthio(MeS—), ethylthio, isopropylthio, etc.].

The term “alkylamino” refers to straight or branched alkylamines of the number of carbon atoms specified (e.g., C1-6 alkylamino), or any number within this range[i.e., methylamino, ethylamino, isopropylamino, t-butylamino, etc.].

The term “alkylsulfonyl” refers to straight or branched chain alkylsulfones of the number of carbon atoms specified (e.g., C1-6 alkylsulfonyl), or any number within this range[i.e., methylsulfonyl(MeSO₂⁻), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range[i.e., methylsulfinyl(MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range[i.e., methyloxycarbonyl(MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5 (6H)-one-3-yl, and the like.

“Heteroaryl” means an aromatic or partially aromatic heterocycle that contains at least one ring heteroatom selected from O, S and N. Heteroaryls thus include heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl(in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

The term «composition» as used herein is intended to encompass a product comprising the specified ingredients in the specified amounts, as well as any product which results, directly or indirectly, from combination of the specified ingredients in the specified amounts. Such term in relation to pharmaceutical composition is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof.

The term «ROR inverse agonist» as used herein is intended to encompass an agent that interacts with the same receptor as an agonist of that receptor, but produces the opposite pharmacological effect.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the inverse agonists of RORs compounds, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the inverse agonists of RORs compounds. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

### Salts and Formulations

It will be understood that, as used herein, references to the inverse agonists of RORs compounds are meant to also include the pharmaceutically acceptable salts, and also salts that are not pharmaceutically acceptable when they are used as precursors to the free compounds or their pharmaceutically acceptable salts or in other synthetic manipulations. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural Formula I are included in the present invention as well.

According to an embodiment, the compounds of structural Formula I may be included in various formulations for use as medicaments. Formulations for oral use may be presented as hard gelatin capsules wherein the active ingredient is mixed with an inert solid diluent, for example, calcium carbonate, calcium phosphate or kaolin, or as soft gelatin capsules wherein the active ingredients is mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methylcellulose, hydroxypropylmethy-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomiser (preferably an I atomiser using electrohydrodynamics to produce a fine mist), or nebuliser, with or without the use of a suitable propellant, such as 1, 1,1,2-tetrafluoroethane or 1,1,1,2,3,3,3-heptafluoropropane. For intranasal use, the powder may comprise a bioadhesive agent, for example, chitosan or cyclodextrin.

The pressurized container, pump, spray, atomizer, or nebuliser contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns).

This may be achieved by any appropriate comminuting method, such as spiral jet milling, fluid bed jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from log to 20 mg of the compound of the invention per actuation and the actuation volume may vary from 11 to 1001. A typical formulation may comprise a compound of formula I, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 ng to 10 mg of the inverse agonists of RORs compounds. The overall daily dose will typically be in the range 1 ng to 10 mg which may be administered in a single dose or, more usually, as divided doses throughout the day.

All the molecules are effective starting at nano molar concentrations and they don't cause cell death in culture in high micro-molar concentrations.

Inverse agonists of RORs compounds may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the inverse agonists of RORs compounds are employed. (For purposes of this application, topical application shall include mouth washes and gargles.)

### Utilities

The compounds specifically exemplified herein exhibit good efficacy in modulating RORγt, as shown by their in vitro assays.

According to an embodiment, the inhibitors of RORγt may improve and may have utility in preventing or treating autoimmune diseases.

One aspect of the invention provides a method for the treatment and control of cancer, which comprises administering to a patient in need of such treatment a therapeutically effective amount of an inverse agonist of RORs compound, and an anticancer agent.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

### Combination Therapy

A patient in need of immunotherapy may be treated with APCs activated with an inverse agonist of RORs compound contemporaneously with other treatments known to the medical practitioner. The use of such multiple treatments may be particularly advantageous to the patient. Such treatments may include, but are not limited to, surgical resection, radiation, chemotherapy, targeted therapy and other types of immunotherapy. Chemotherapy agents that may be used include:
a) cytotoxic agents such as taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof;
b) antimetabolites such as methotrexate, 6-mercaptopurine, 6-thioguanine, cytarabine, 5-fluorouracil decarbazine;
c) alkylating agents such as mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin;
d) anthracyclines such as daunorubicin and doxorubicin;
e) antibiotics such as dactinomycin, bleomycin, mithramycin, and anthramycin (AMC);
f) anti-mitotic agents such as vincristine and vinblastine;
g) targeted therapies that may be used include, but they are not limited to: hormone therapies (such as degarelix an luteinizing hormone-releasing hormone (LHRH) antagonist that reduces testosterone levels in prostate cancer), signal transduction inhibitors (such as imatinib and trastuzumab), as well as gene expression modulators (for example the HDAC inhibitors panobinostat and belinostat), apoptosis inducers (such as recombinant human TNF-related apoptosis-inducing ligand (TRAIL)) and angiogenesis inhibitors (such as sorafenib, sunitinib, pazopanib and everolimus);
h) Immunotherapy agents that may be used include: monoclonal antibodies treatment (anti-CTLA4, anti-PD1), and chimeric antigen receptors (CARs)-T-Cells.

### Assays for Measuring Biological Activity

Activity of the compounds of this application may be evaluated using the following assays for RORγt-inhibiting activity. Compounds of Formula I will have activities of <10 μM in this assay, and preferably, activity of <1 μM.

PBMCs were treated with CD3/CD28 for 14 days in presence of IL-6 10 ng/ml, IL-1B 10 ng/ml, TGF-B 10 ng/ml, and IL-23 10 ng/ml.

Culture medium was changed every other day for the whole duration.

Drugs were added to the polarized cells at day 14 for 48 hours.

PMA/lonomycin/Monensin cocktail was added for 6 hours to the cells at the end of 48 hours incubation.

Live/dead (455 UV), cell surface (CD4, CD3, CD8), and intracellular nuclear staining (IL-17, IL-21, IL-22 and ROR gamma)

### 1) Mouse Syngeneic Tumor Model

A novel dual reporter (GFP & luciferase) MSCV based plasmid was creating by subcloning from two commercially available constructs (addgene 19360: pLenti pgk and addgene 18751: MSCV-IRES-GFP). This construct was used to generate retrovirus for infection of EG7 lymphoma cells (ATCC #CRL-2133). EG7-ova expressing cells are a commonly used model for T cell lymphoma engineered to express the ovalbumin antigen making them useful in immunological and drug discovery applications. These cells were then GFP sorted at the McGill University Life Sciences Complex flow cytometry core (BD FACscalibur) prior to subcutaneous implantation (5×10⁵ cells) in syngeneic C57bl6 mice (Harlan). Tumors were allowed to establish in the mice and then were imaged ten days post implantation (Perkin Elmer IVIS100) and luminescence was quantified using IVIS software. Mice were then treated on day 10 post injection with 2×10⁶ ex vivo-derived and inhibitor-treated mature dendritic cells (IP injection) (see ex vivo activation of monocyte derived DC with PTP inhibitor). Ovalbulim was added to DC cultures during maturation at a concentration of 2.5 mg/mL. Following maturation, DCs were harvested by gently resuspending with phosphate-buffered saline to dislodge any loosely attached cells. Cells were then spun down and washed twice with phosphate-buffered saline. Prior to injection, DCs were resuspended in phosphate buffered saline at a concentration of 20 million cells per mL.

### Preparation of Compounds of the Invention

The novel compounds of Formula I can be synthesized from the available building blocks by the methods of Combinatorial chemistry. The synthetic accessibility score of the novel compounds described in the invention are ranging from 2-5.

The definition of Synthetic accessibility score is

Synthetic accessibility score: from 1 (very easy) to 10 (very difficult) based on 1024 fragmental contributions (FP2) modulated by size and complexity penaties, trained on 12′782′590 molecules and tested on 40 external molecules (r2=0.94).

Combinatorial chemistry comprises chemical synthetic methods that make it possible to prepare a large number (tens to thousands or even millions) of compounds in a single process. These compound libraries can be made as mixtures, sets of individual compounds or chemical structures generated by computer software. Combinatorial chemistry can be used for the synthesis of small molecules and for peptides.

Strategies that allow identification of useful components of the libraries are also part of combinatorial chemistry. The methods used in combinatorial chemistry are applied outside chemistry, too.

In the drug discovery process, the synthesis and biological evaluation of small molecules of interest has typically been a long and laborious process. Combinatorial chemistry has emerged in recent decades as an approach to quickly and efficiently synthesize large numbers of potential small molecule drug candidates. In a typical synthesis, only a single target molecule is produced at the end of a synthetic scheme, with each step in a synthesis producing only a single product. In a combinatorial synthesis, when using only single starting material, it is possible to synthesize a large library of molecules using identical reaction conditions that can then be screened for their biological activity. This pool of products is then split into three equal portions containing each of the three products, and then each of the three individual pools is then reacted with another unit of reagent B, C, or D, producing 9 unique compounds from the previous 3. This process is then repeated until the desired number of building blocks is added, generating many compounds. When synthesizing a library of compounds by a multi-step synthesis, efficient reaction methods must be employed, and if traditional purification methods are used after each reaction step, yields and efficiency will suffer.

Solid-phase synthesis offers potential solutions to obviate the need for typical quenching and purification steps often used in synthetic chemistry. In general, a starting molecule is adhered to a solid support (typically an insoluble polymer), then additional reactions are performed, and the final product is purified and then cleaved from the solid support. Since the molecules of interest are attached to a solid support, it is possible to reduce the purification after each reaction to a single filtration/wash step, eliminating the need for tedious liquid-liquid extraction and solvent evaporation steps that most synthetic chemistry involves. Furthermore, by using heterogeneous reactants, excess reagents can be used to drive sluggish reactions to completion, which can further improve yields. Excess reagents can simply be washed away without the need for additional purification steps such as chromatography.

Over the years, a variety of methods have been developed to refine the use of solid-phase organic synthesis in combinatorial chemistry, including efforts to increase the ease of synthesis and purification, as well as non-traditional methods to characterize intermediate products. Although the majority of the examples described here will employ heterogeneous reaction media in every reaction step, Booth and Hodges provide an early example of using solid-supported reagents only during the purification step of traditional solution-phase syntheses. In their view, solution-phase chemistry offers the advantages of avoiding attachment and cleavage reactions necessary to anchor and remove molecules to resins as well as eliminating the need to recreate solid-phase analogues of established solution-phase reactions.

The single purification step at the end of a synthesis allows one or more impurities to be removed, assuming the chemical structure of the offending impurity is known. While the use of solid-supported reagents greatly simplifies the synthesis of compounds, many combinatorial syntheses require multiple steps, each of which still requires some form of purification. Armstrong, et al. describe a one-pot method for generating combinatorial libraries, called multiple-component condensations (MCCs). In this scheme, three or more reagents react such that each reagent is incorporated into the final product in a single step, eliminating the need for a multi-step synthesis that involves many purification steps. In MCCs, there is no deconvolution required to determine which compounds are biologically-active because each synthesis in an array has only a single product, thus the identity of the compound should be unequivocally known.

In another array synthesis, Still generated a large library of oligopeptides by split synthesis. The drawback to making many thousands of compounds is that it is difficult to determine the structure of the formed compounds. Their solution is to use molecular tags, where a tiny amount (1 μmol/bead) of a dye is attached to the beads, and the identity of a certain bead can be determined by analyzing which tags are present on the bead. Despite how easy attaching tags makes identification of receptors, it would be quite impossible to individually screen each compound for its receptor binding ability, so a dye was attached to each receptor, such that only those receptors that bind to their substrate produce a color change.

When many reactions need to be run in an array (such as the 96 reactions described in one of Armstrong's MCC arrays), some of the more tedious aspects of synthesis can be automated to improve efficiency. DeWitt and Czarnik detail a method called the “DIVERSOMER method,” in which many miniaturized versions of chemical reactions are all run simultaneously. This method uses a device that automates the resin loading and wash cycles, as well as the reaction cycle monitoring and purification, and demonstrate the feasibility of their method and apparatus by using it to synthesize a variety of molecule classes, such as hydantoins and benzodiazepines, running 40 individual reactions in most cases.

Oftentimes, it is not possible to use expensive equipment, and Schwabacher, et al. describe a simple method of combining parallel synthesis of library members and evaluation of entire libraries of compounds. In their method, a thread that is partitioned into different regions is wrapped around a cylinder, where a different reagent is then coupled to each region which bears only a single species. The thread is then re-divided and wrapped around a cylinder of a different size, and this process is then repeated. The beauty of this method is that the identity of each product can be known simply by its location along the thread, and the corresponding biological activity is identified by Fourier transformation of fluorescence signals.

In most of the syntheses described here, it is necessary to attach and remove the starting reagent to/from a solid support. This can lead to generation of a hydroxyl group, which can potentially affect the biological activity of a target compound. Ellman uses solid phase supports in a multi-step synthesis scheme to obtain 192 individual 1,4-benzodiazepine derivatives, which are well-known therapeutic agents. To eliminate the possibility of potential hydroxyl group interference, a novel method using silyl-aryl chemistry is used to link the molecules to the solid support which cleaves from the support and leaves no trace of the linker.

When anchoring a molecule to a solid support, intermediates cannot be isolated from one another without cleaving the molecule from the resin. Since many of the traditional characterization techniques used to track reaction progress and confirm product structure are solution-based, different techniques must be used. Gel-phase 13 C NMR spectroscopy, MALDI mass spectrometry, and IR spectroscopy have been used to confirm structure and monitor the progress of solid-phase reactions. Gordon et al., describe several case studies that utilize imines and peptidyl phosphonates to generate combinatorial libraries of small molecules. To generate the imine library, an amino acid tethered to a resin is reacted in the present of an aldehyde. The authors demonstrate the use of fast 13 C gel phase NMR spectroscopy and magic angle spinning 1 H NMR spectroscopy to monitor the progress of reactions and showed that most imines could be formed in as little as 10 minutes at room temperature when trimethyl orthoformate was used as the solvent. The formed imines were then derivatized to generate 4-thiazolidinones, B-lactams, and pyrrolidines.

The use of solid-phase supports greatly simplifies the synthesis of large combinatorial libraries of compounds. This is done by anchoring a starting material to a solid support and then running subsequent reactions until a sufficiently large library is built, after which the products are cleaved from the support. The use of solid-phase purification has also been demonstrated for use in solution-phase synthesis schemes in conjunction with standard liquid-liquid extraction purification techniques.

The following Examples are provided to illustrate the invention and are not to be construed as limiting the invention in any manner. The scope of the invention is defined by the appended claims.

### Example 1

### Patient Data

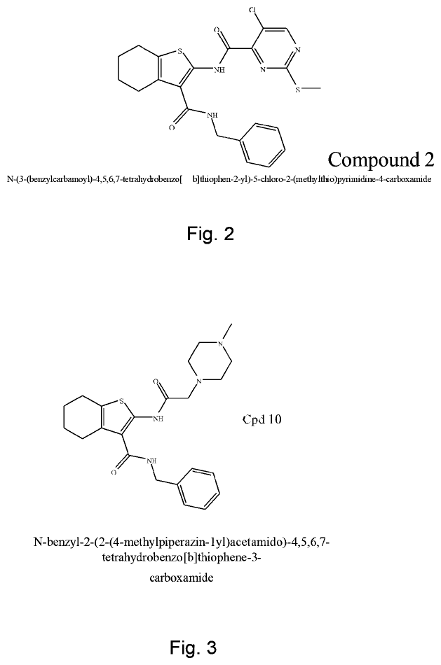

Compounds shown in FIGS. 2 and 3 were used.

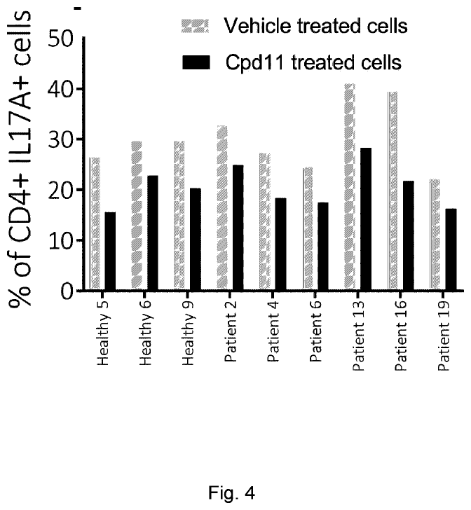

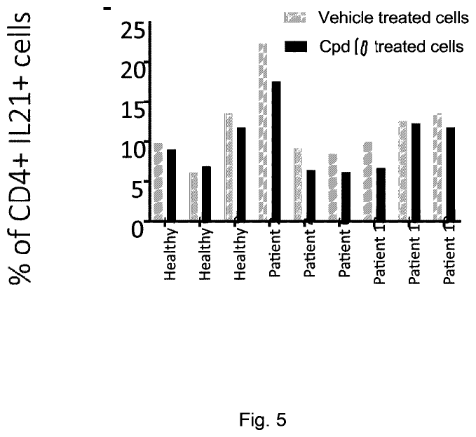

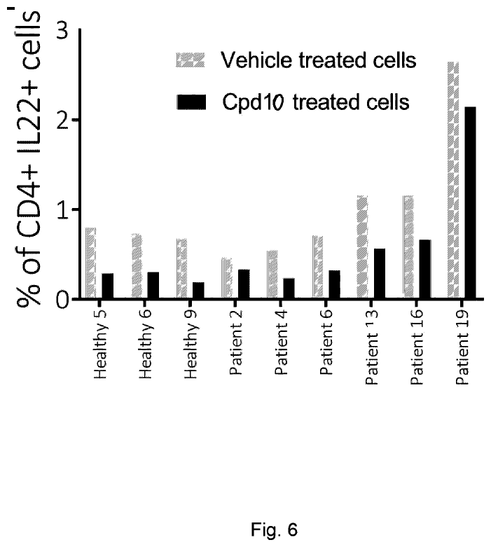

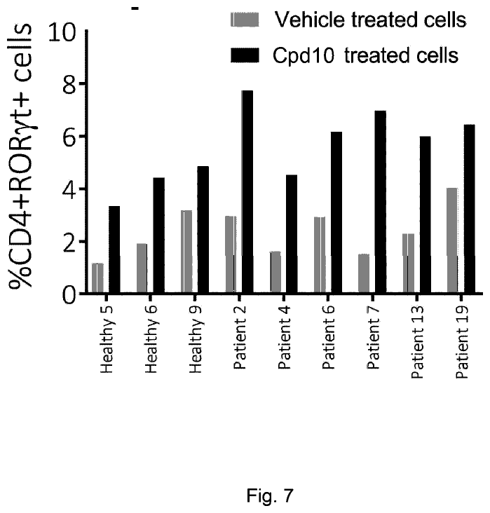

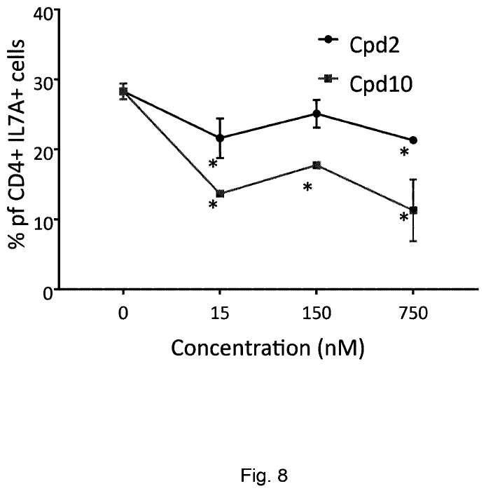

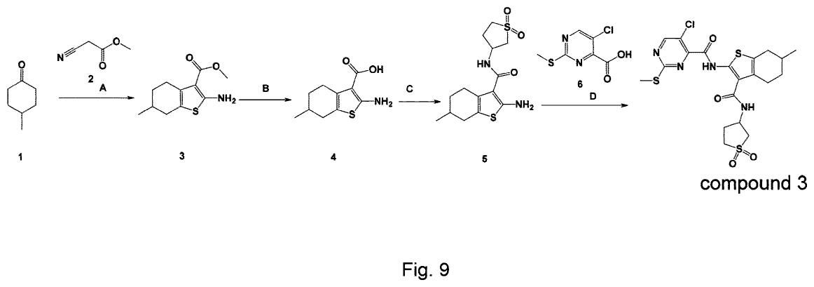

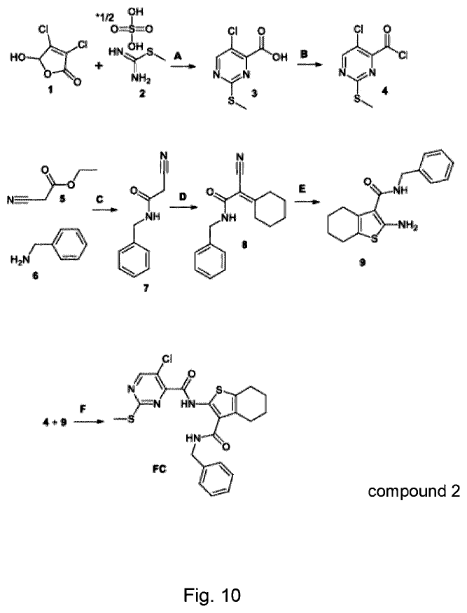

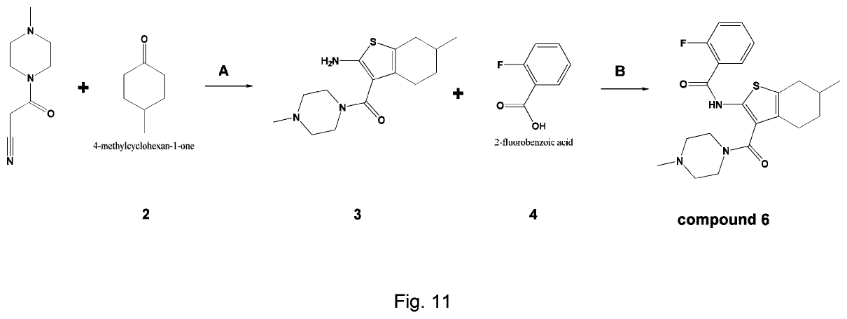

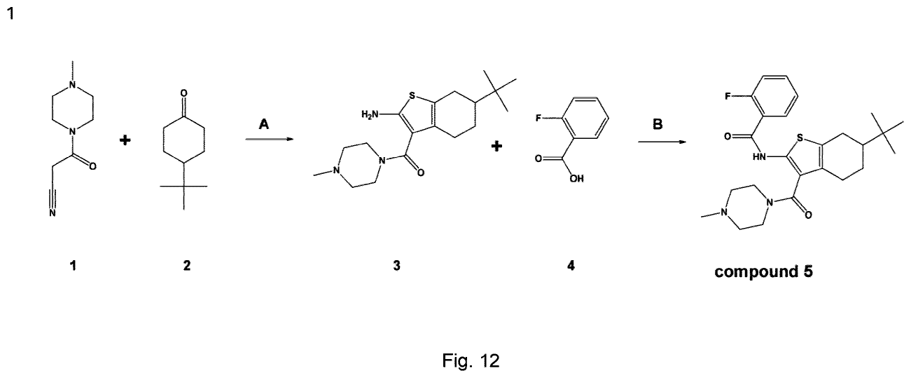

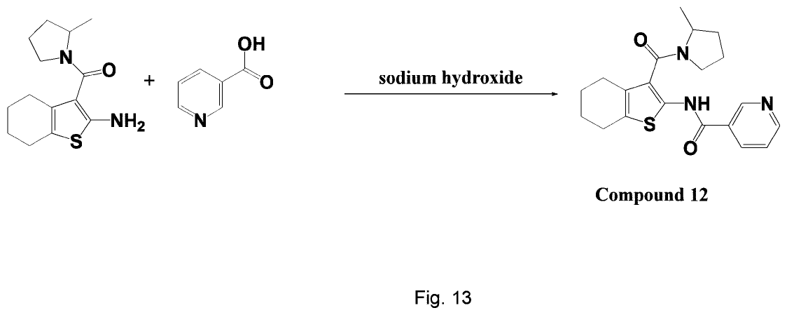

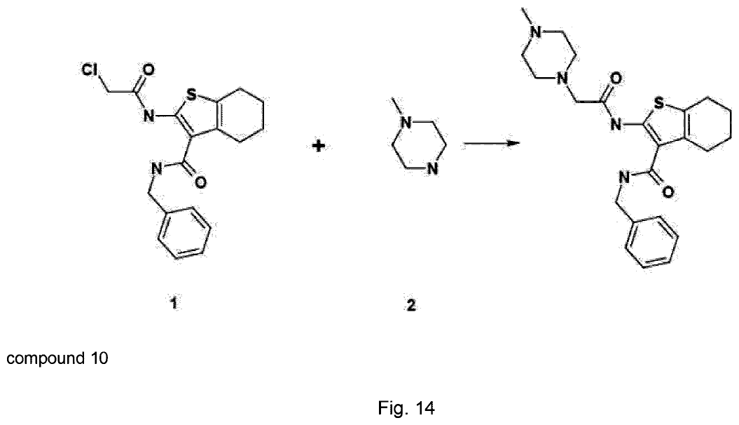

Results are shown in FIGS. 4 to 8.

Patients 2, 7, 13 and 16 have calculated panel reactive antibodies (cPRA)>80%.

Patients 4, 6 and 19 have calculated panel reactive antibodies (cPRA)<20%.

Healthy stands for healthy volunteers cPRA not known and the PBMCs were used as control.

Patients with cPRA>80% are known as sensitized patients or highly sensitized patients and they develop an immunologic rejection of solid organ grafts.

### Definition of Calculated Panel Reactive Antibodies (cPRA)

A panel-reactive antibody (PRA) is an antibody that is reactive against any of several known specific antigens in a test panel. A panel-reactive antibody test (PRA test) is an immunologic test routinely performed by clinical laboratories on the blood of people awaiting organ transplantation. The PRA score is expressed as a percentage between 0% and 100%. It represents the proportion of the population to which the person being tested will react via pre-existing antibodies against human antigens, especially HLA system antigens. These antibodies target the surface antigens of target cells, such as HLAs. In other words, it is a test of the degree of alloimmunity in a graft recipient and thus a test that quantifies the risk of transplant rejection. Each population will have a different demographic prevalence of particular HLA antigens, and so the PRA test panel constituents will differ from country to country. A high PRA value usually means that the individual is primed to react immunologically against a large proportion of the population. Individuals with a high PRA value are often termed “sensitized”, which indicates that they have been exposed to “foreign” (or “non-self”) proteins in the past and have developed antibodies to them. These antibodies develop following previous transplants, blood transfusions and pregnancy. Transplanting organs into recipients who are “sensitized” to the organs significantly increases the risk of rejection, resulting in higher immunosuppressant requirement and shorter transplant survival. People with high PRA scores therefore spend longer waiting for an organ to which they have no pre-existing antibodies. Extensive efforts have been made to identify treatment regimes to reduce PRA in sensitized transplant candidates. In certain circumstances, plasma exchange, intravenous immunoglobulin, rituximab and other “antibody-directed” immune therapies may be employed, but this is an area in which active investigation continues.

### Example 2

### Novel Th17 Inhibitors to Treat Lethal Acute Respiratory Distress Syndrome in Covid-19

The main cause of death as a result of the COVID-19 infection is lung inflammation. This problem, known as ARDS, is the result of an exceptionally high release of cytokine proteins, one of the most important of which is IL-17. This cytokine initiates inflammation and recruits other white blood cells, with the purpose of fighting infection. An abnormally excessive response results in tissue damage, ARDS and death. We have developed new drugs that reduce the production of IL-17 and may prevent ARDS and death. We propose to test these drugs in the cells from COVID-19 patients and in a model of IL-17 mediated lung inflammation to see if this can prevent death due to the complications of COVID-19.

Acute respiratory distress syndrome (ARDS) is the predominant cause of mortality in patients with COVID-19 (1). ARDS is thought to involve responses driven by type 3 innate lymphoid cells (ILC3) and Th17 lymphocytes (2-4). ILC3 and Th17 cells have pro-inflammatory roles, which include recruitment of neutrophils and release of IL-17, granulocyte-macrophage colony stimulating factor and IL-22 (5,6). Their identity is defined by the expression of the transcription factor, retinoic acid receptor-related orphan receptor gamma, RORγt (7). RORγt is a key transcription factor in the induction of Th17-specific genes, including IL17A/F, IL21, IL22, IL23R, CCR4, and CCR6.

Considerable evidence suggests a role for Th17 and ILC3 in the development of ARDS in COVID-19. IL-17 is a key cytokine in LPS induced acute lung injury (ALI) and ARDS (4,8), and it is seen as early as 2 days post-infection with influenza, prior to neutrophil recruitment (9). Antibodies to IL-17, and IL-17 receptor antagonists, both ameliorate ALI (9,10). In human studies, elevations in circulating and alveolar IL-17A are associated with increased alveolar neutrophil infiltration, as well as measures of inflammation and organ dysfunction in ARDS (3). Identical IL-17 gene polymorphisms confer a risk for both allergic asthma (11) as well as ARDS (12). Enhancement of IL-17 has previously been observed in infection with SARSCOV (13,14) and MERS-COV (15,16), as well as H1N1 influenza (17). Early evidence supports a similar pathogenesis in COVID-19 (1). Therefore, the Th17 axis forms an early response to viral respiratory infection, upstream of neutrophil recruitment, and those with exaggerated Th17 cytokine responses may be at particular risk of developing ARDS.

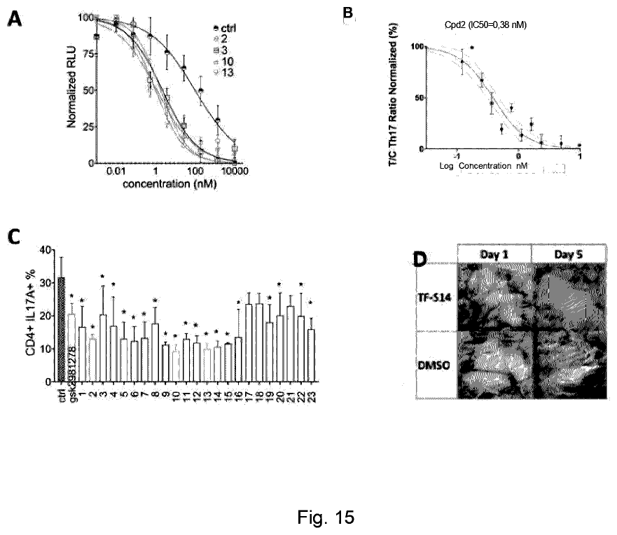

Using in silico drug design methods, we have developed 23 novel small molecules that inhibit RORγt. We have demonstrated their ability to suppress Th17 activation in cells from allosensitized renal transplant candidates. Time-resolved fluorescence energy transfer assays were performed to evaluate the binding specificities and IC50 of the compounds compared with GSK2981278 (GSK) (a known RORγt inhibitor). RORγt co-activator recruitment was <1 μM for all the compounds tested (FIG. 15A). Th17 polarization in cell preparations was effectively inhibited with IC50's in the nM range (FIG. 15B). Several of our novel compounds (grey bars) compared favourably with GSK (black bar) (FIG. 15C). Non-Th17 cells (Th1, Th2 and Treg) were unaffected.

We then assessed the in vivo efficacy of these molecules using a pre-sensitized skin allograft model (BALB/c to C57BL/6), in which graft rejection is rapid (<5 days) and a Th17 response is prominent. Our compounds were highly effective at prolonging allogeneic skin graft survival with no observed toxicity. Epidermal hyperplasia, loss of skin appendages (hair follicles, sebaceous glands) and extensive dermal inflammation were present in DMSO controls and absent in treated recipients (1 mg/kg) (FIG. 15D). Pathological findings confirm blockade of early Th17 inflammation.

### Example 3

### Rorc in Pancreatic Cancer

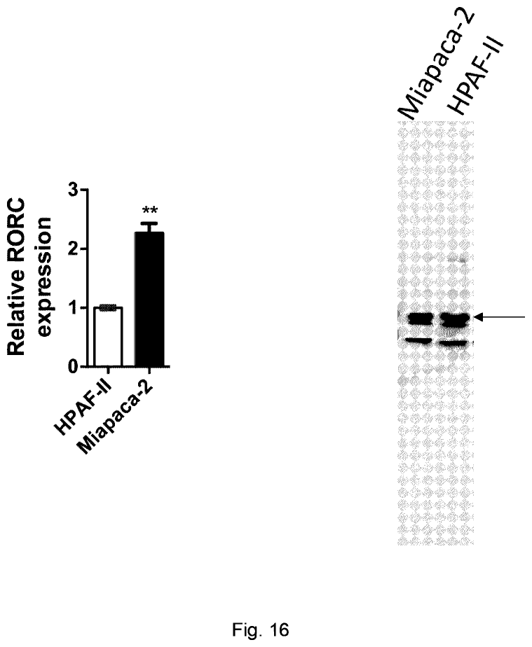

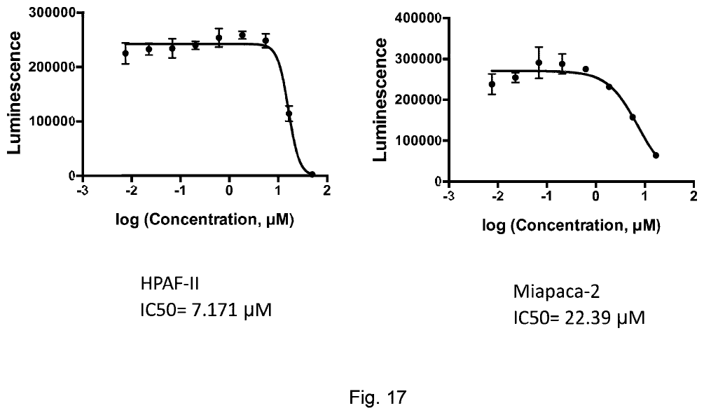

FIG. 16 shows western presence of RORC in 2 human pancreatic cell lines. FIG. 17 shows viability using compound 10 for each cell line.

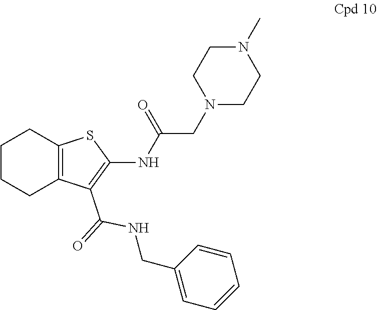 [SMILES: CN1CCN(CC(=O)Nc2sc3c(c2C(=O)NCc2ccccc2)CCCC3)CC1]

N-benzyl-2-(2-(4-methylpiperazin-1-yl) acetamido)-4,5,6,7-tetrahydrobenzo[b]thiophene-3-carboxamide

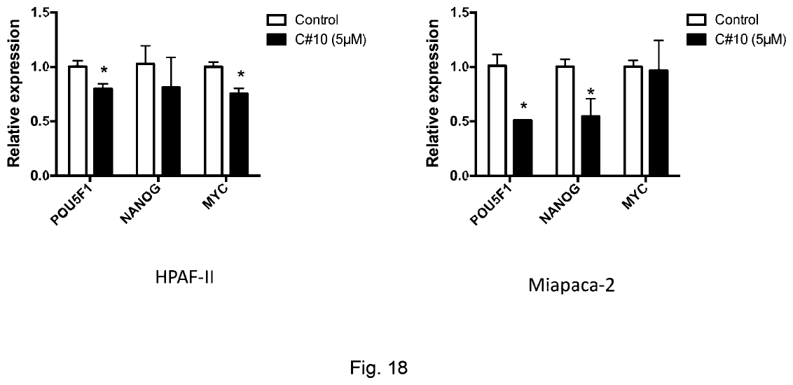

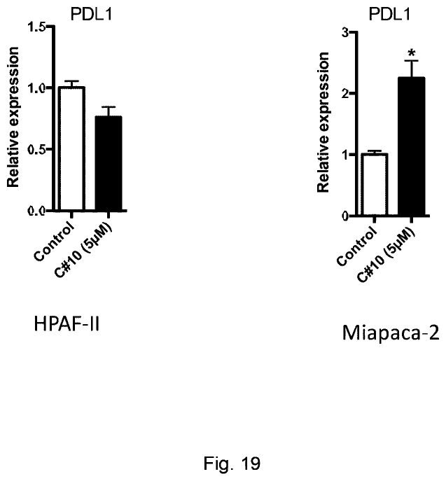

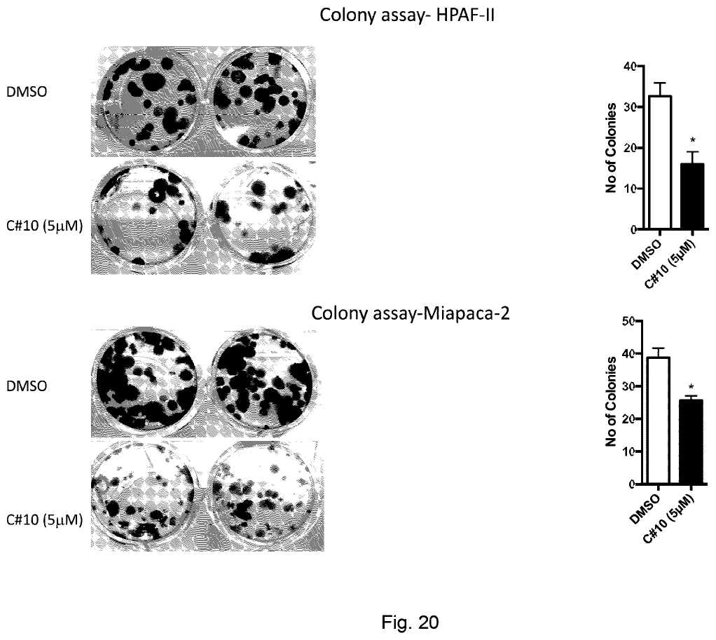

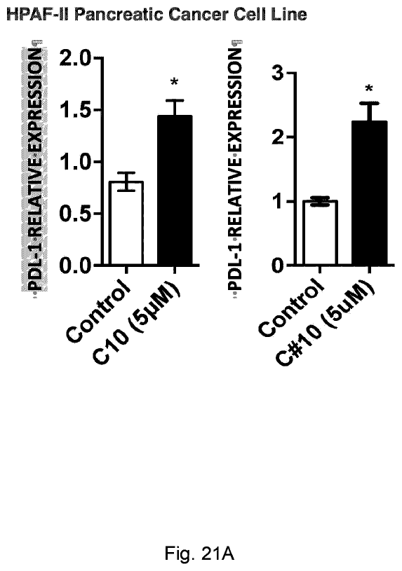

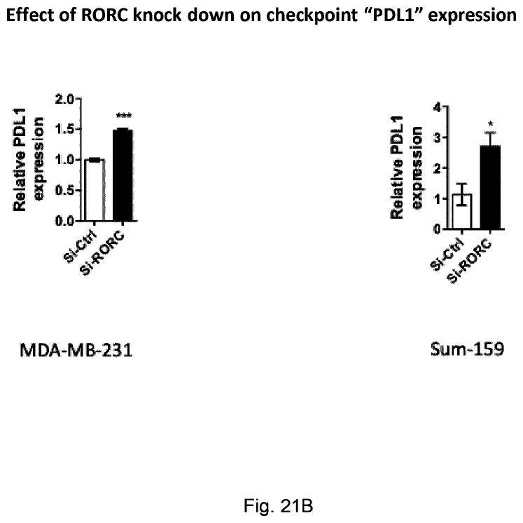

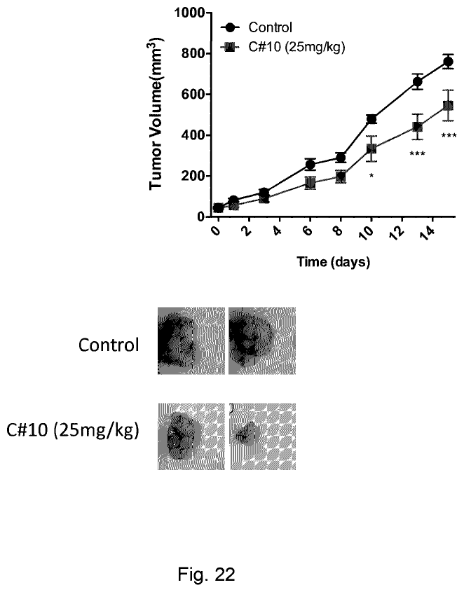

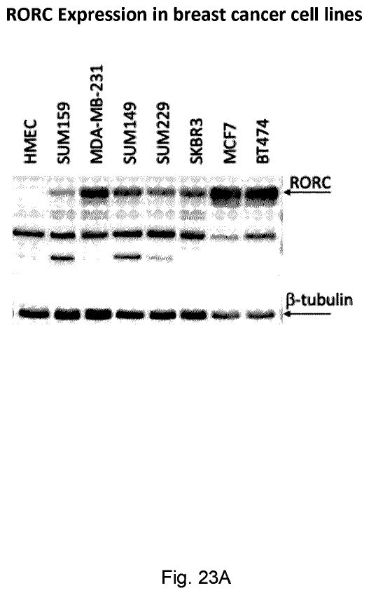

FIG. 18 shows the effect of compound 10 on stem cell markers at 5 mM which is still very low concentrations. FIG. 19 shows the effect of compound 10 on expression of the immune checkpoint PDL1 in pancreatic cancer which is relatively low. Compound 10 increases that expression and could be used in synergy with PDL1 inhibitors (immune checkpoint inhibitors like Pembrolizumab and Nivolumab). FIG. 20 shows the effect of compound 10 on colony forming assays. FIGS. 21A & 21B shows effect of compound 10 on stem cells as they express ALDH activity. Increase in PDL-1 expression with the addition of compound 10 indicates the cancer is less able to remain ‘stealth’ (remain hidden) from the patient's immune system. FIG. 22 shows the effect of compound 10 on pancreatic cancer growth with HPAF cell line in NSG mice on cancer volume. NSG mice have attenuated immune system so they are used as a model for growing Human cancer cell lines.

### Example 4

### Rorc in Breast Cancer

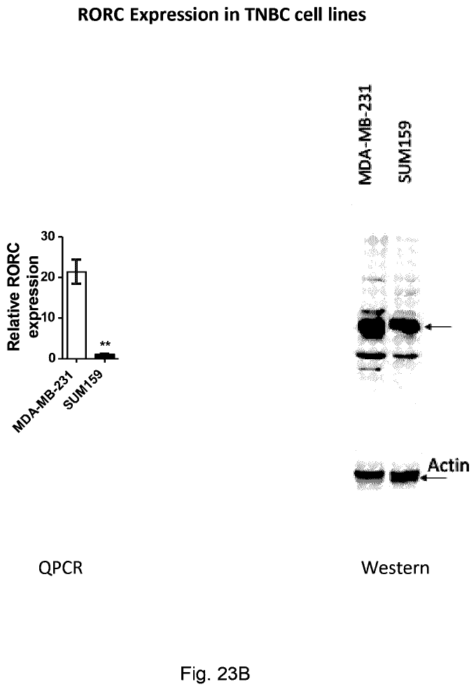

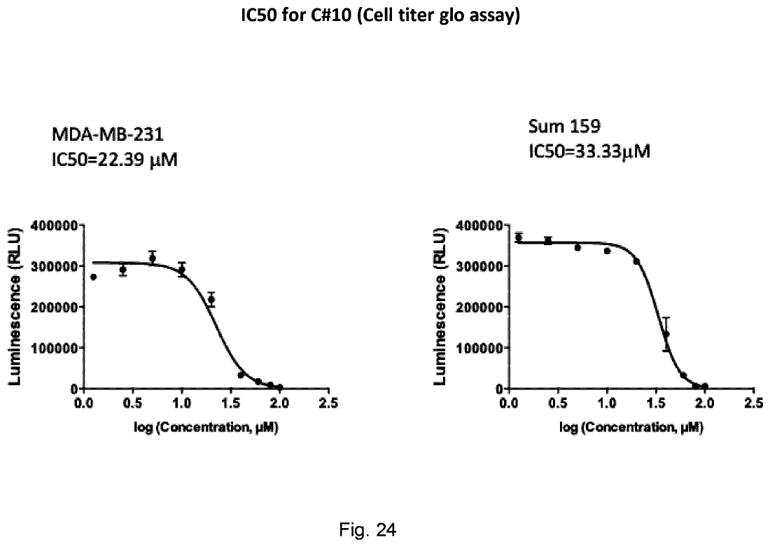

FIGS. 23A and 23B shows western presence of RORC in 2 human breast cell lines. FIG. 24 shows viability using compound 10 for each cell line.

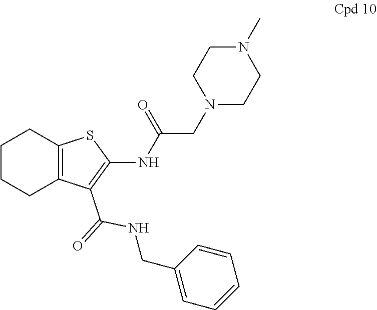 [SMILES: CN1CCN(CC(=O)Nc2sc3c(c2C(=O)NCc2ccccc2)CCCC3)CC1]

N-benzyl-2-(2-(4-methylpiperazin-1-yl) acetamido)-4,5,6,7-tetrahydrobenzo[b]thiophene-3-carboxamide

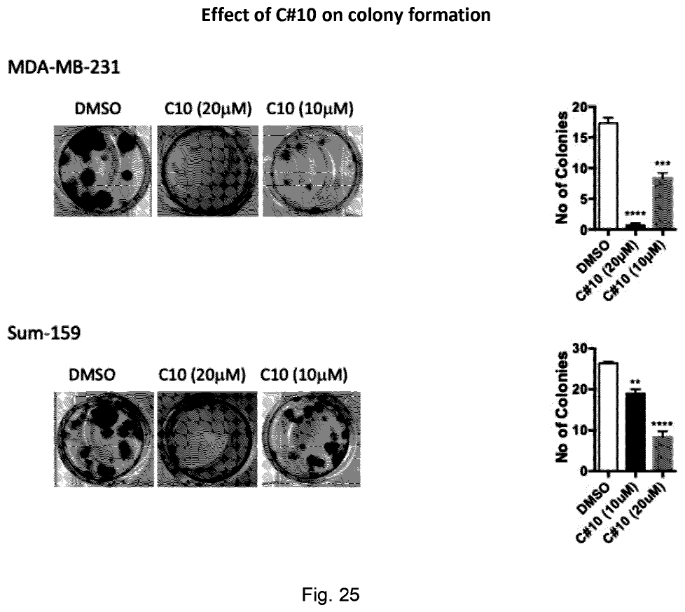

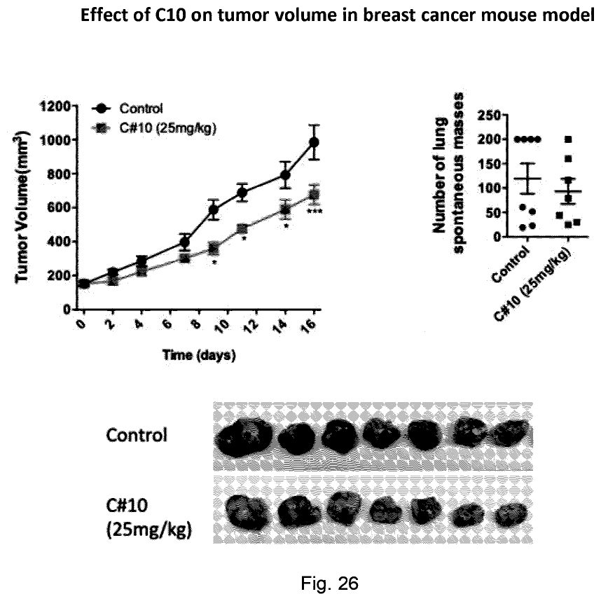

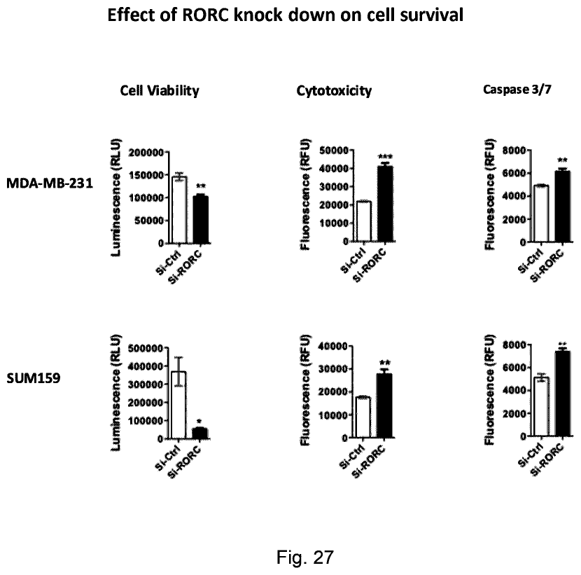

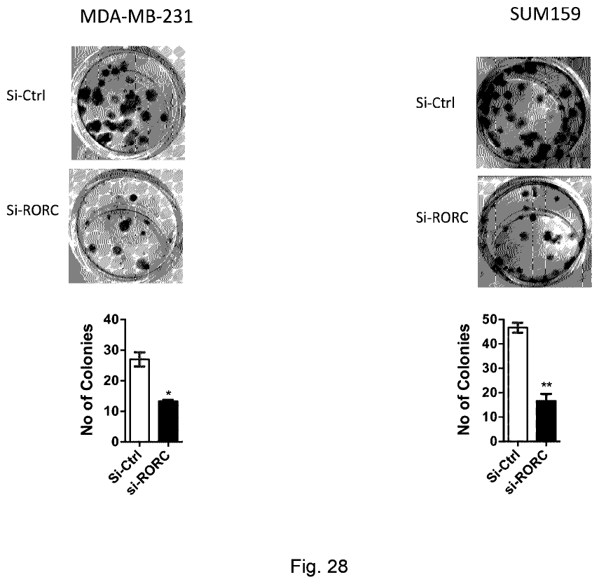

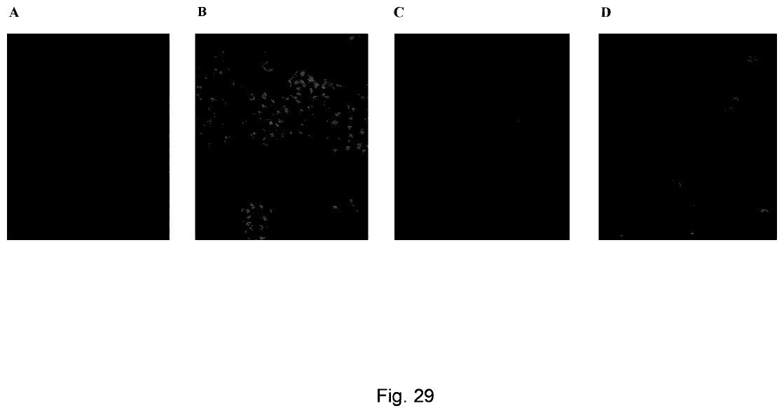

FIG. 25 illustrates the effect of 10 and 20 micromolar of compound 10 on Colony assay-MDA and on Colony assay-SUM. FIG. 26 illustrates the effect of 25 mg/kg daily injections of compound 10 on tumour volume in a NSG mouse model. The tumours are allowed to grow to about a volume between 150-200 mm³ and then compound given daily for 2 weeks. FIG. 27 illustrates the relative silencing of RORC on cell viability, cytotoxicity, and Caspase 3/7 a marker of Apoptosis. FIG. 29 illustrates the effect of 5 micromolar on Cell colony forming units. Compound 10 significantly reduces the ability to form Colony forming units.

### Example 5

### HCC Data with Human HuH-7 and Mouse Hepa1-6

FIG. 29 illustrates in vitro immunofluorescence expression of RORy (A) and IL17 (B). HCC cell lines HuH-7 and Hepa1-6 express nuclear receptor RORγ. Human HCC cell line HuH-7 and mouse HCC cell line Hepa1-6 were stained for RORγ and IL-17 and expression was visualized via immunofluorescence microscopy. A) RORγ expression in HuH-7. RORγ in red and nucleus of cells in blue. B) IL-17 expression in HuH-7. IL-17 in red and nucleus of cells in blue. C) RORγ expression in Hepa1-6. RORγ in red and nucleus of cells in blue. D) IL-17 expression in Hepa1-6. IL-17 in red and nucleus of cells in blue.

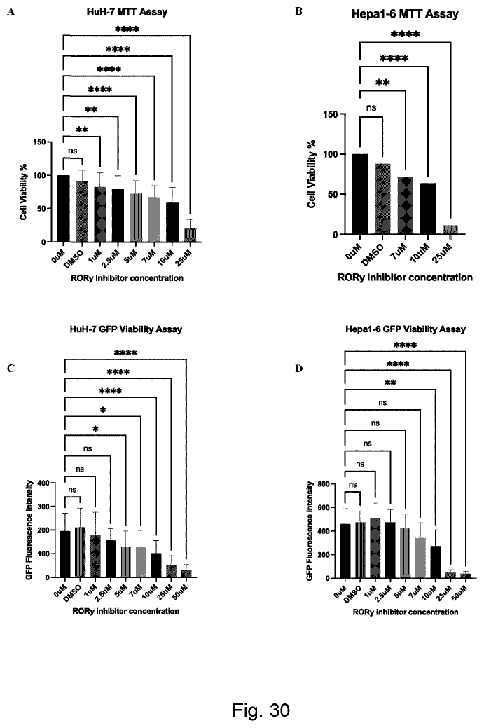

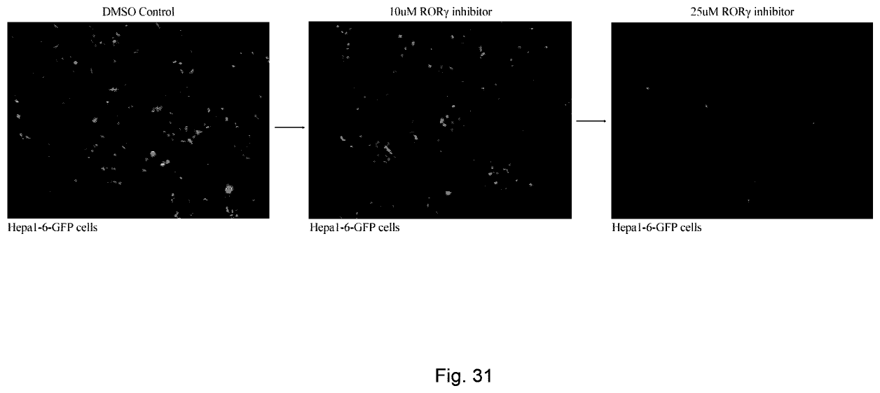

FIG. 30 illustrates in vitro cell viability assay both human HuH-7 and mouse Hepa1-6 cell lines with increasing concentrations of compound 10. In the human cell line you see an effect with as low as 1mMol concentration. HuH-7 and Hepa1-6 cell viability assays. A) HuH-7 MTT assay reveals a significant and dose dependent difference in cell viability when subjected to varying concentrations of RORγ inhibitor C#10. B) Hepa1-6 MTT assay reveals a significant and dose dependent difference in cell viability when subjected to varying concentrations of RORγ inhibitor C#10. C) HuH-7 GFP viability assay reveals a significant and dose dependent difference in cell viability when subjected to varying concentrations of RORγ inhibitor C#10. D) Hepa1-6 GFP viability assay reveals a significant and dose dependent difference in cell viability when subjected to varying concentrations of RORγ inhibitor C#10.

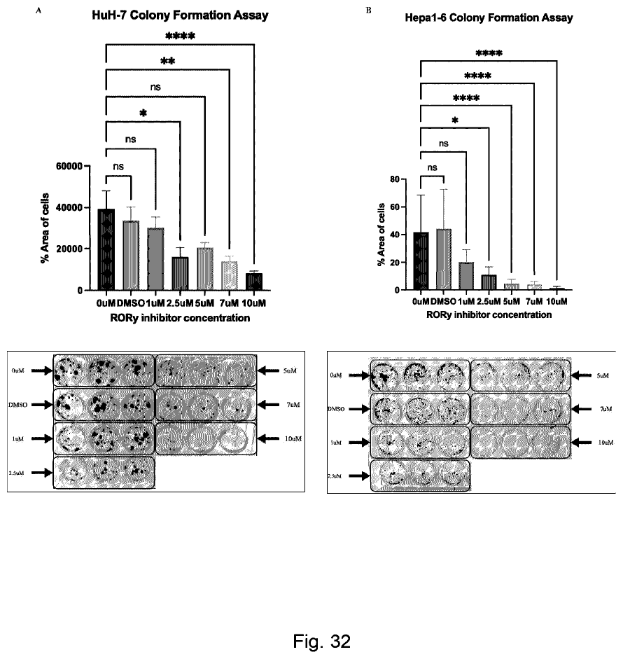

FIG. 32 illustrates in vitro cell viability using another assay GFP (part of FIG. 30 in a bar graph).

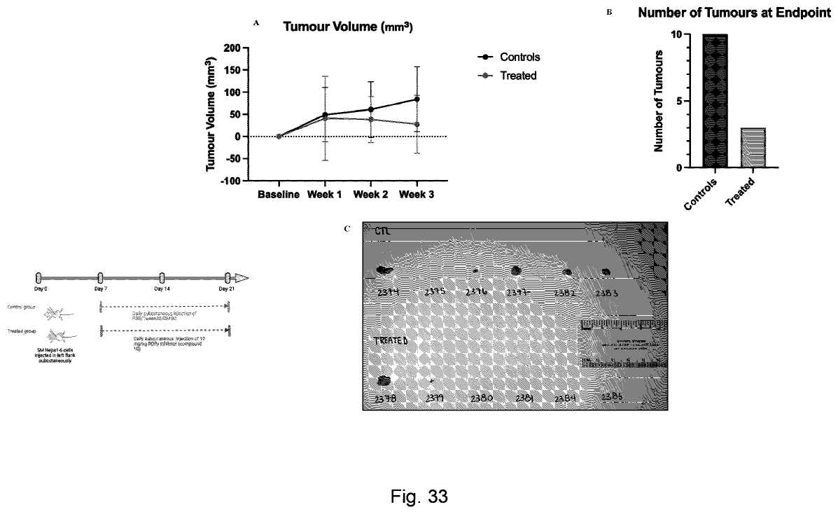

FIG. 33 illustrates another in vitro viability assay Colony Formation Assay. We show that both cell lines are suppressed at 2.5 mMol concentration. HuH-7 and Hepa1-6 colony formation assays. A) HuH-7 colony formation assay reveals a significant and dose dependent difference in colony growth ability when subjected to varying concentrations of RORγ inhibitor C#10. B) Hepa1-6 colony formation assay reveals a significant and dose dependent difference in colony growth ability when subjected to varying concentrations of RORy inhibitor C#10.

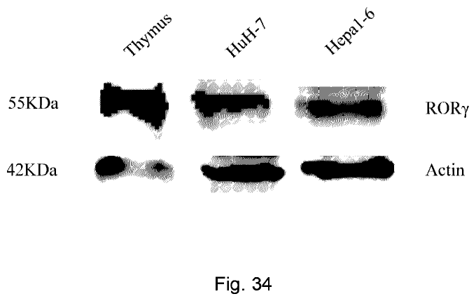

FIG. 34 illustrates the effect of the compound in an in vivo mouse model of HCC injected in the flank. In many of the treated mice (over 3 weeks) no cancer is seen in most of the treated mice. HCC mouse model. Hepa1-6 cells (5×10⁶) were suspended in 100 μl of PBS and injected into the left flank of C57BL6 mice subcutaneously with 1 ml syringe. Mice were treated with a daily dose of RORγ inhibitor for 14 days. Subsequently, tumours were resected and tumour volume was measured. A) A decrease in tumour volume is visible over the course of 21 days in treated groups of mice compared to the control group. B) Total number of tumours at endpoint of experiment in control and treated groups. C) Representative pictures of tumours in control and treated groups at endpoint.

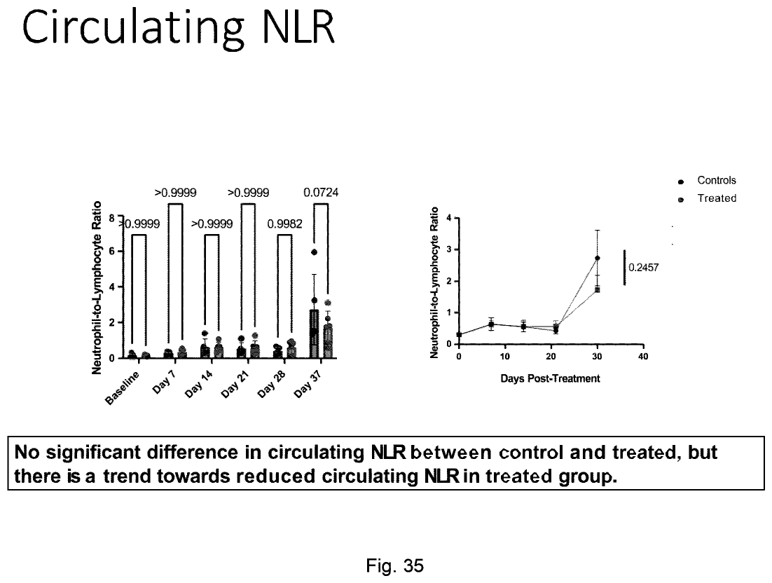

FIG. 35 illustrates Western blot analysis reveals RORy expression in HuH-7 and Hepa1-6 cell lines. Western blot showing expression of RORγ (~55 kDa) in HuH-7 and Hepa1-6 cell lines. Mouse Thymus was used as a positive control. Actin (~42 kDa), was used as a control for protein loading and fraction purity.

### Example 6

### Assess the In Vivo Therapeutic Potential of RORy in Gastric Cancer Model

### Material and Method

5M YTN16-GFP cells injected in flank of C57BL/6 mice
Control group
Treated group-25 mg/kg RORy inhibitor
   Inhibitor=experimental compound obtained from Dr Tchervenkov's lab

Daily s.c injections of the drug start once tumour is palpable
Same for all mice.

Treatment is continued till tumour size reaches around 1.5 cm³ among most mice.

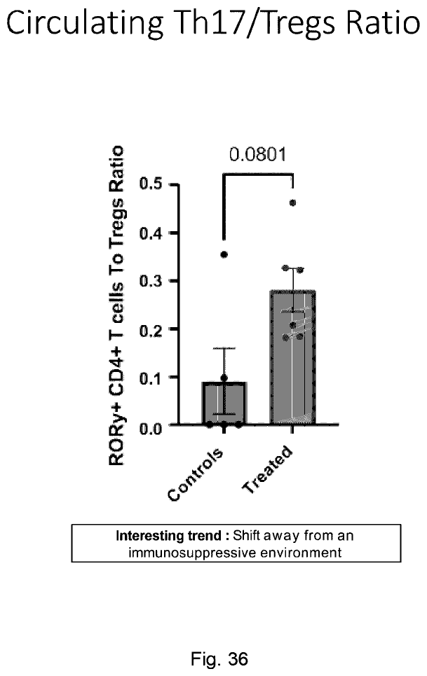

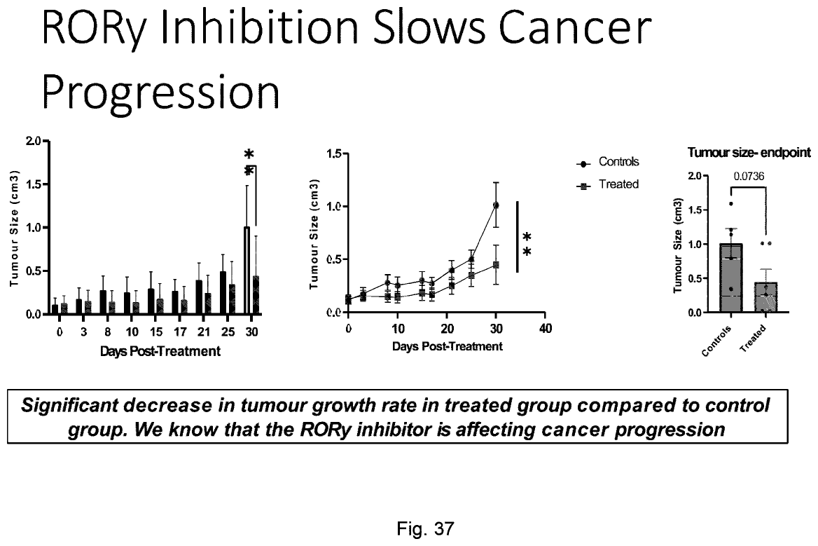

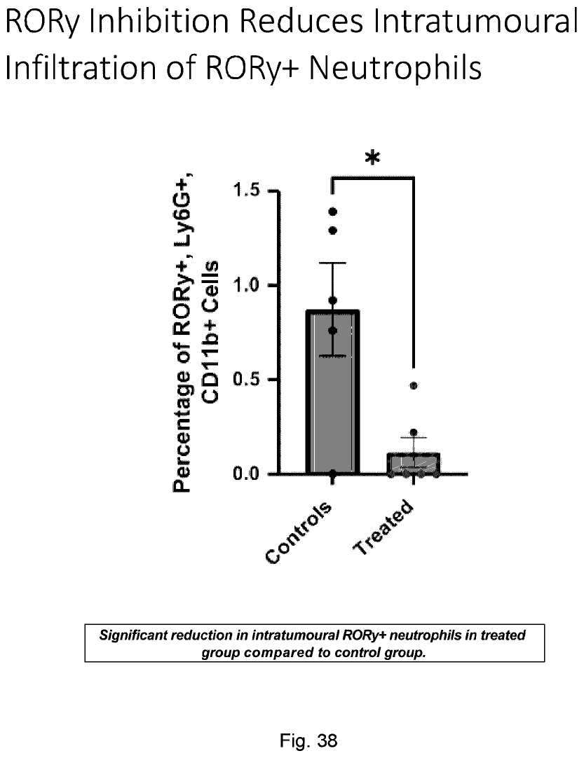

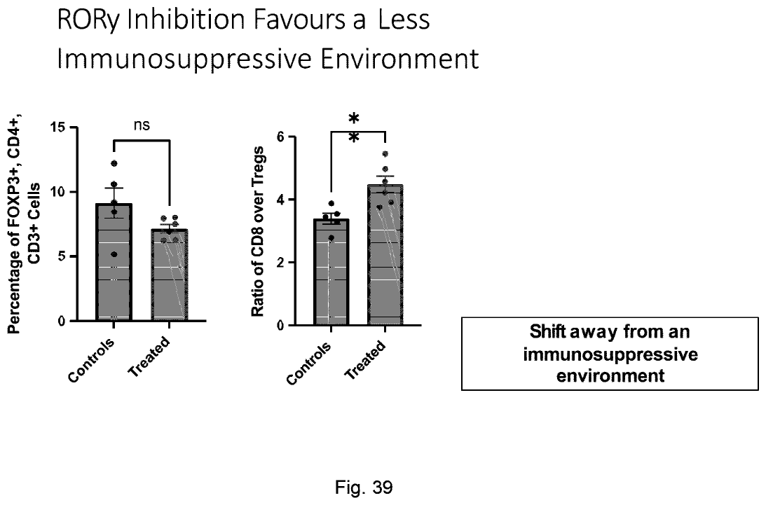

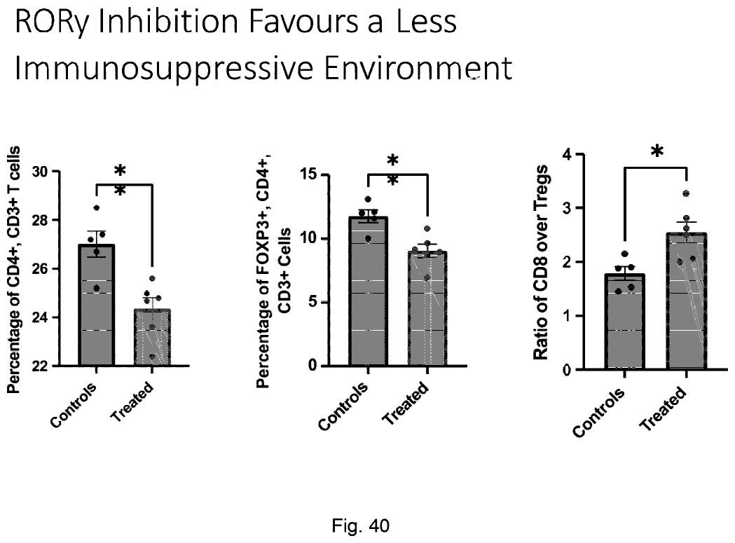

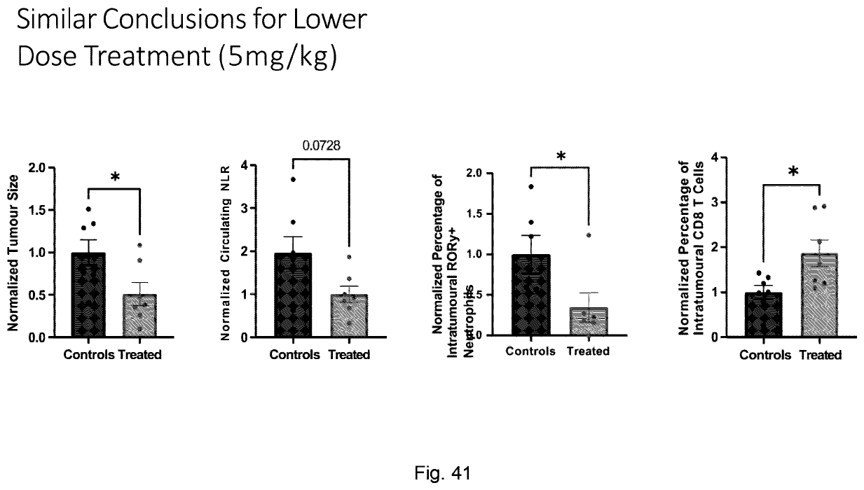

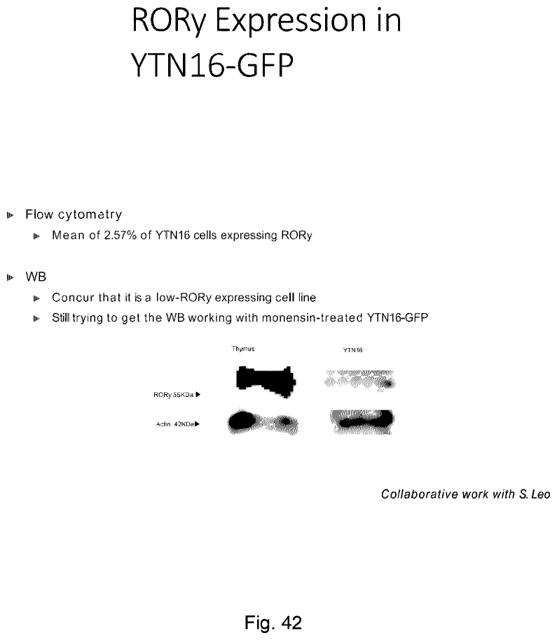

Results are shown in FIGS. 35 to 42.

### Conclusion

Daily RORy inhibition in a murine gastric cancer model slows down tumour growth.

Within the circulation, there is a small trend of shifting away from an immunosuppressive microenvironment. However, the treatment did not have any significant effect on the circulating immune population.

Within the tumour and the BM, we again observed the shift away from an immunosuppressive phenotype. We also see reduced intratumoural infiltration of RORγ+ neutrophils.

Same conclusions reached when experiment was done with a lower dose (5 mg/kg)-done 2×.

### Example 7

### Quantify Prevalence of RORγ+ Neutrophils in Blood and in Tumor of GEA Patients

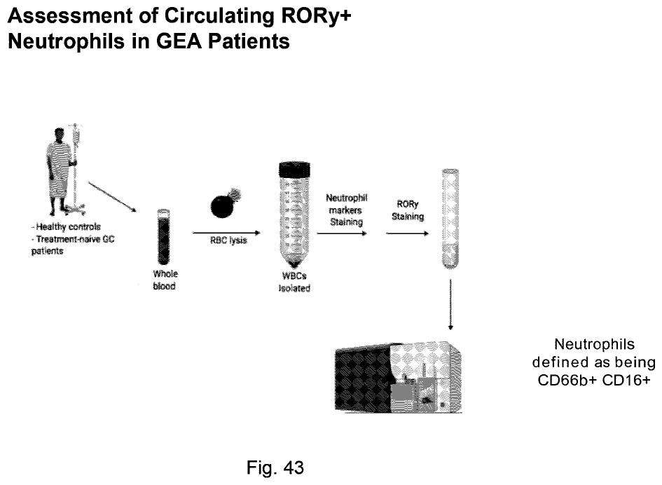

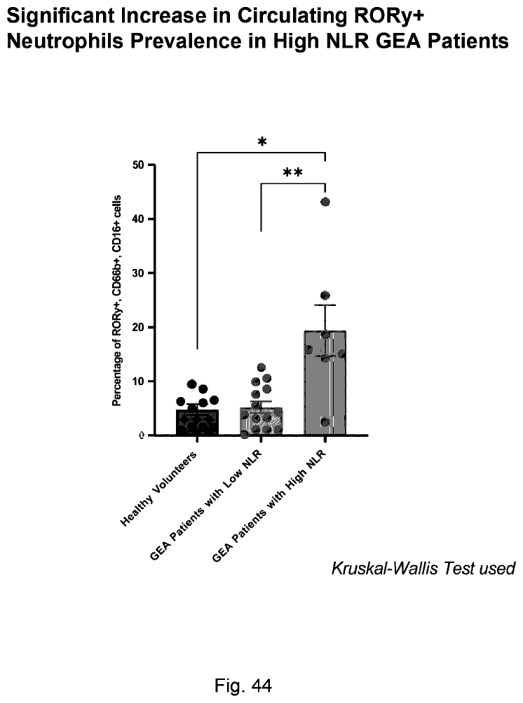

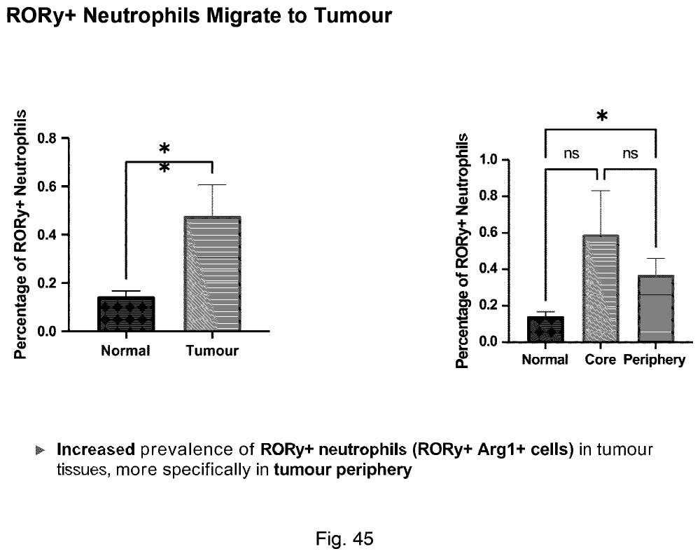

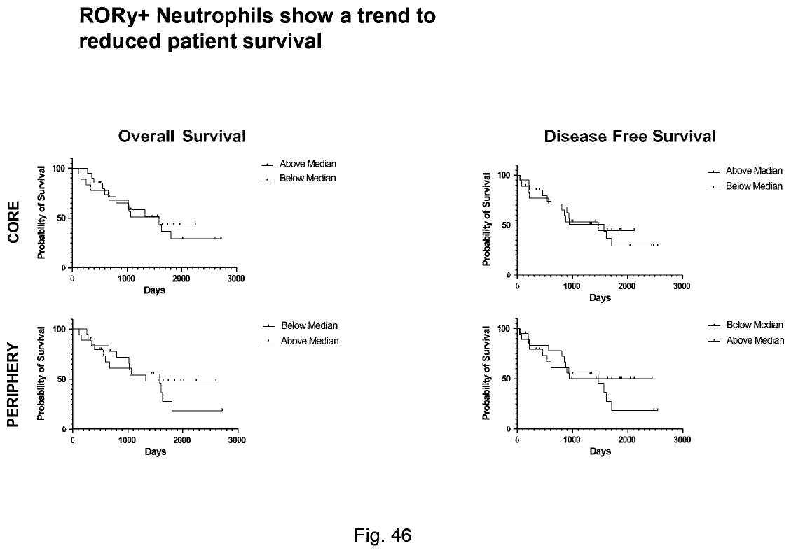

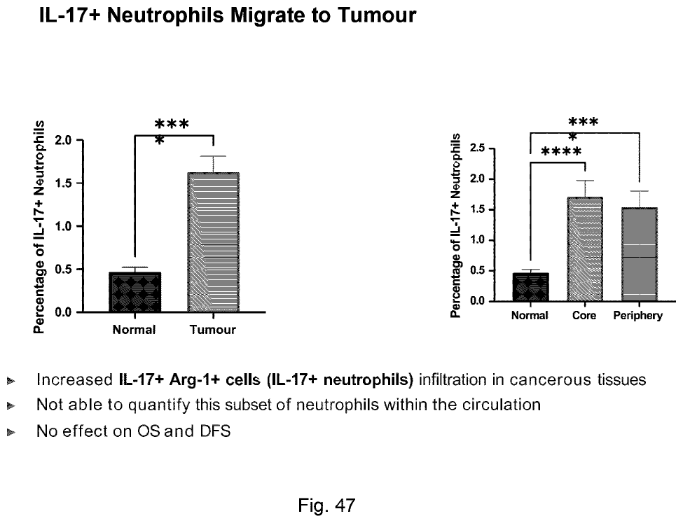

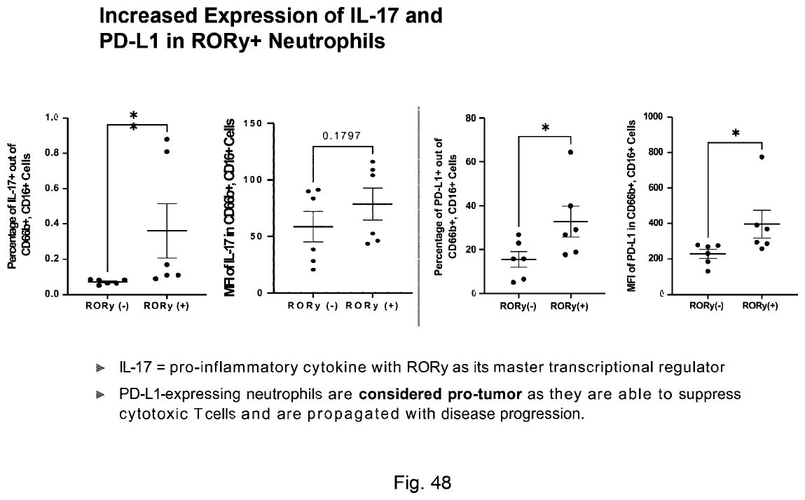

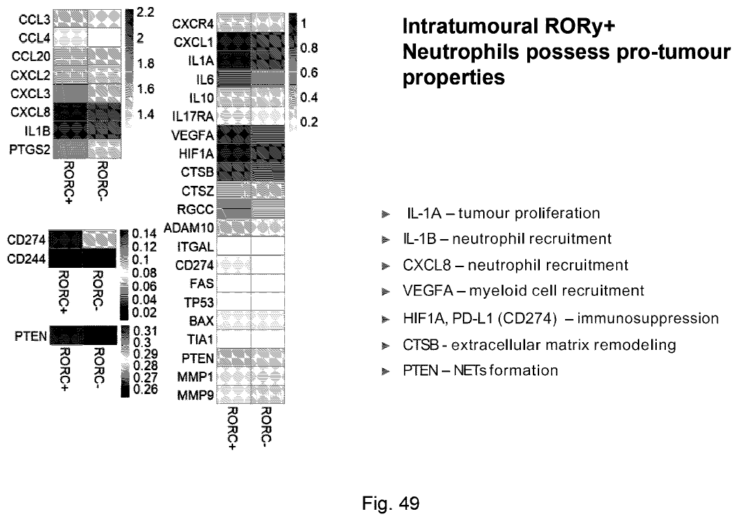

Results are shown in FIGS. 43 to 49.

### Example 8

### Identification of Neutrophils that Inhibit Response to Cancer Therapy

Julia Kargl et al. (JCI Insight. 2019; 4 (24): e130850) showed that in patients treated with Immune checkpoint inhibitors PD1 inhibitors, the presence of high intra-cancer neutrophils and a high ratio of neutrophils to CD8+ lymphocytes in the cancer is predictive of either no response or progression of cancer under PD1 immunotherapy. In a mouse model when they combined an anti-neutrophil and inhibitors, the cancer responded to treatment with immunotherapy anti PD1 inhibitor.

In accordance with the present invention, we further define these neutrophils that inhibit response to cancer therapy and behave in suppressing delivery to cancers of CD8+ lymphocytes as being these RORC+ Neutrophils.

### Example 9

### Balb/c Skin Graft Survival in Sensitized C57BL/6 Recipients

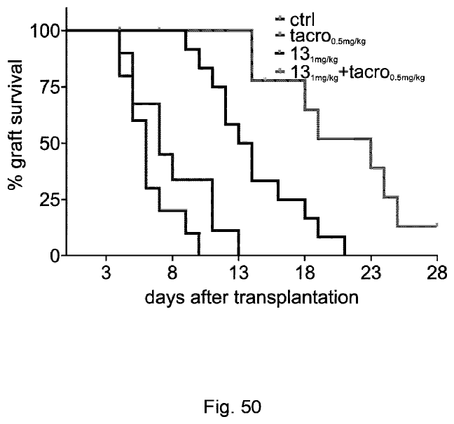

C57BL/6 mice were sensitized by 3 IP injections of 10 M Balb/c splenocytes at day 0, 7 and 14. The mice were transplanted at day 15 with 1 cm² skin grafts from Balb/c mice and treated daily with ip injections of vehicle (ctrl), cpd 13 1 mg/kg (13_{1 mg/kg}), tacrolimus 0.5 mg/kg (tacro_{0.5 mg/kg}) and cpd13 1 mg/kg+tacrolimus 0.5 mg/kg (13_{1 mg/kg}+tacro_{0.5 mg/kg}). P-value<<0.001, mantel-cox test; median graft survival: ctrl=6 days, tacro_{0.5 mg/kg}=7 days, 13_{1 mg/kg}=13.5 days and cpd13 13_{1 mg/kg}+tacro_{0.5 mg/kg}=23 days. Results are shown in FIG. 50.

### Example 10

### Single Cell RNA Sequencing of Triple Negative Breast Cancer with RORC Inverse Agonist

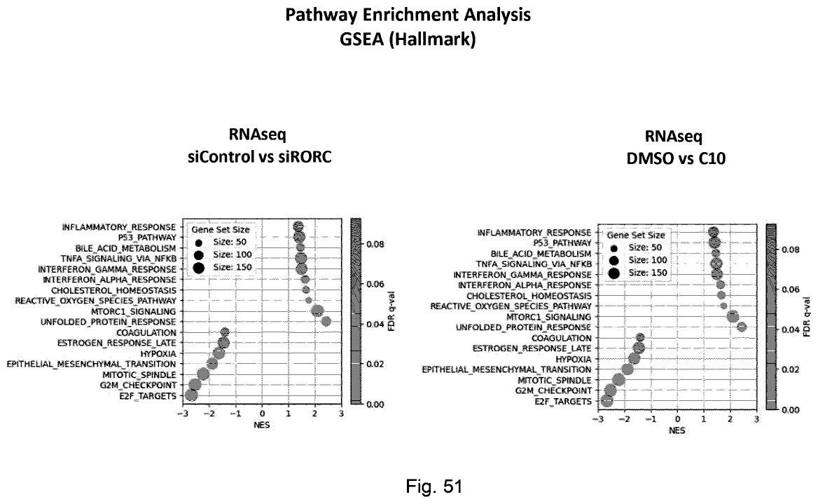

FIG. 51 illustrates that RORC inhibition is associated with upregulation of inflammatory response, increases in p53 pathway, increases in TNFa signaling via NFKb increases interferon gamma and interferon response. It is also associated with a decreased late estrogen response, decreased hypoxia response, decreased epithelial mesenchymal transition, decreased mitotic spindle, reduction in G2M checkpoint and E2F targets.

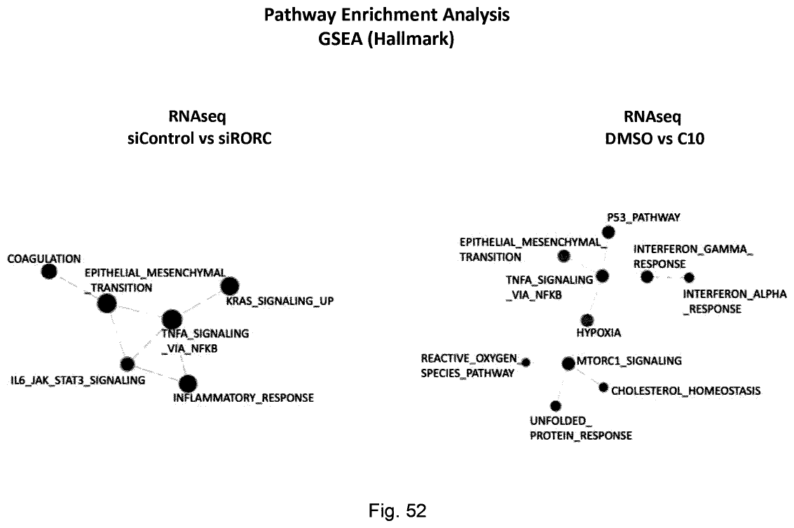

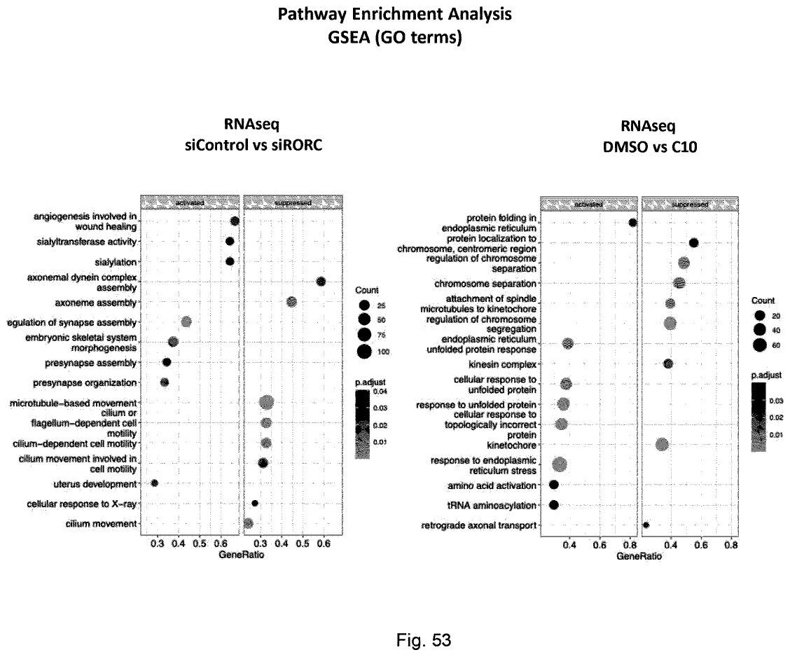

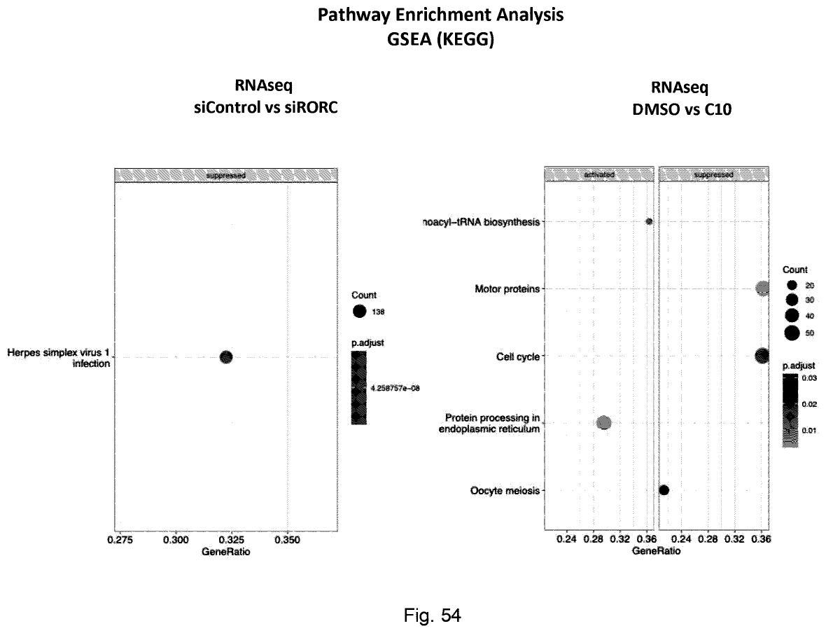

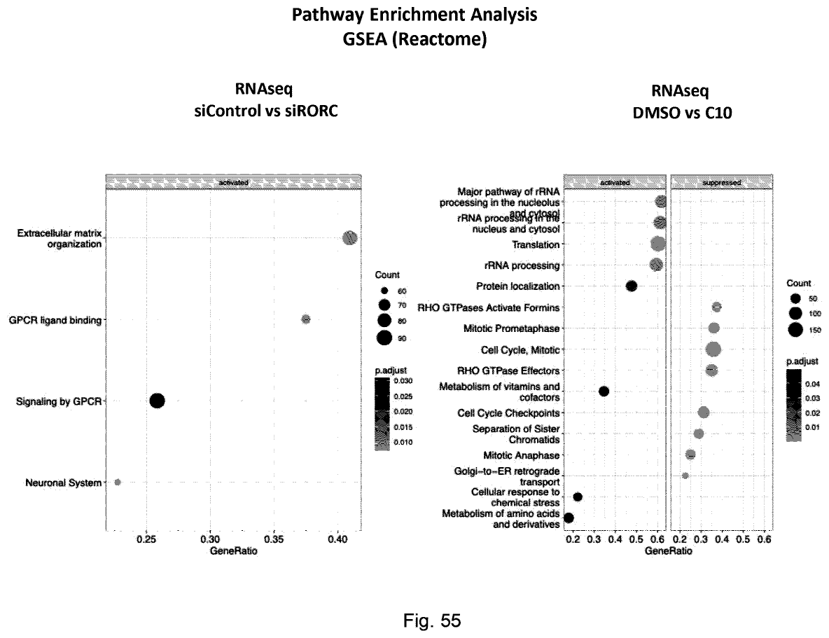

FIGS. 52-55 illustrate the ability of RORgamma inverse agonists to suppress the ability of cancer cells to undergo mitosis, mitotic cell cycle, separation of sister chromatid and mitotic anaphase and chromosome separation, all indicating a total arrest of cancer cell division. In addition it suppresses microtubule-based movement, cillium or flagellium-dependent cell motility, cilium movement involved in cell motility, all indicating a total arrest the ability of cancer cell to migrate and metastasize.

While preferred embodiments have been described above and illustrated in the accompanying drawings, it will be evident to those skilled in the art that modifications may be made without departing from this disclosure. Such modifications are considered as possible variants comprised in the scope of the disclosure.

### REFERENCES

1. Xu Z, Shi L, Wang Y, Zhang J, Huang L, Zhang C, et al. Pathological findings of COVID-19 associated with acute respiratory distress syndrome. Lancet Respir Med. 2020 Feb. 18.
2. Ardain A, Porterfield J Z, K I verpris H N, Leslie A. Type 3 ILCs in Lung Disease. Front Immunol. Frontiers; 2019; 10:92.
3. Mikacenic C, Hansen E E, Radella F, Gharib S A, Stapleton R D, Wurfel M M. Interleukin-17A Is Associated With Alveolar Inflammation and Poor Outcomes in Acute Respiratory Distress Syndrome. Crit Care Med. 2016 March; 44 (3): 496-502.
4. Li J T, Melton A C, Su G, Hamm D E, LaFemina M, Howard J, et al. Unexpected Role for Adaptive αβTh17 Cells in Acute Respiratory Distress Syndrome. The Journal of Immunology. American Association of Immunologists; 2015 Jul. 1; 195 (1): 87-95.
5. Buonocore S, Ahern P P, Uhlig H H, Ivanov I I, Littman D R, Maloy K J, et al. Innate lymphoid cells drive interleukin-23-dependent innate intestinal pathology. Nature. 2010 Apr. 29; 464 (7293): 1371-5.
6. Xu S, Cao X. Interleukin-17 and its expanding biological functions. Cell Mol Immunol. Nature Publishing Group; 2010 May; 7 (3): 164-74.
7. Ivanov II, Mckenzie B S, Zhou L, Tadokoro C E, Lepelley A, Lafaille J J, et al. The orphan nuclear receptor RORgammat directs the differentiation program of proinflammatory IL-17+ T helper cells. Cell. 2006 Sep. 22; 126 (6): 1121-33.
8. Ding Q, Liu G-Q, Zeng Y-Y, Zhu J-J, Liu Z-Y, Zhang X, et al. Role of IL-17 in LPS induced acute lung injury: an in vivo study. Oncotarget. 2017 Nov. 7; 8 (55): 93704-11.
9. Crowe C R, Chen K, Pociask D A, Alcorn J F, Krivich C, Enelow R I, et al. Critical role of IL-17RA in immunopathology of influenza infection. The Journal of Immunology. American Association of Immunologists; 2009 Oct. 15; 183 (8): 5301-10.
10. Li C, Yang P, Sun Y, Li T, Wang Z, Zou Z, et al. IL-17 response mediates acute lung injury induced by the 2009 pandemic influenza A (H1N1) virus. Cell Res. Nature Publishing Group; 2012 March; 22 (3): 528-38.
11. Du J, Han J-C, Zhang Y-J, Qi G-B, Li H-B, Zhang Y-J, et al. Single-Nucleotide Polymorphisms of IL-17 Gene Are Associated with Asthma Susceptibility in an Asian Population. Med Sci Monit. 2016 Mar. 8; 22:780-7.
12. Xie M, Cheng B, Ding Y, Wang C, Chen J. Correlations of IL-17 and NF-κB gene polymorphisms with susceptibility and prognosis in acute respiratory distress syndrome in a chinese population. Biosci Rep. 2019 Feb. 28; 39 (2): 860.
13. Nicholls J M, Poon L L M, Lee K C, Ng W F, Lai S T, Leung C Y, et al. Lung pathology of fatal severe acute respiratory syndrome. The Lancet. 2003 May 24; 361 (9371): 1773-8.
14. Wong C K, Lam C W K, Wu A K L, Ip W K, Lee N L S, Chan I H S, et al. Plasma inflammatory cytokines and chemokines in severe acute respiratory syndrome. Clin Exp Immunol. John Wiley & Sons, Ltd; 2004 April; 136 (1): 95-103.
15. Faure E, Poissy J, Goffard A, Fournier C, Kipnis E, Titecat M, et al. Distinct immune response in two MERS-COV-infected patients: can we go from bench to bedside? Baldanti F, editor. PLOS ONE. 2014; 9 (2): e88716.
16. Mahallawi W H, Khabour O F, Zhang Q, Makhdoum H M, Suliman B A. MERS-COV infection in humans is associated with a pro-inflammatory Th1 and Th17 cytokine profile. Cytokine. 2018 April; 104:8-13.
17. Bermejo-Martin J F, Ortiz de Lejarazu R, Pumarola T, Rello J, Almansa R, Ram rez P, et al. Th1 and Th17 hypercytokinemia as early host response signature in severe pandemic influenza. Crit Care. 2009; 13 (6):R201.

1. (canceled)

2. A method of preventing or treating a disease, autoimmune disease or antibody mediated rejection in a patient in a patient in need thereof, wherein said disease is characterized by an elevated neutrophil to lymphocyte ratio (NLR) in the peripheral blood of the patient, which comprises administering to said patient a therapeutically effective amount of a ROR inverse agonist, its pharmaceutically acceptable salt or stereoisomers thereof, wherein said neutrophils express nuclear receptors (RORs) alpha (RORα or RORA) or gamma (RORC, RORy or RORγt).

3. (canceled)

4. The method of claim 2, wherein said disease is HIV, cancer, celiac disease, diabetes mellitus type 1, Graves' disease, inflammatory bowel disease, psoriasis, rheumatoid arthritis, systemic lupus erythematosus, asthma, dermatitis, fatty liver disease, Crohn's disease, cardiovascular disease, inflammatory diseases, neurological disorder, multiple sclerosis, acute respiratory distress syndrome or arteriosclerosis.

5. (canceled)

6. The method of claim 4, wherein said cancer is prostate cancer, breast cancer, ovarian cancer, multiple myeloma, brain cancer, glioma, lung cancer, salivary cancer, stomach cancer, thymic epithelial cancer, thyroid cancer, leukemia, melanoma, lymphoma, gastric cancer, pancreatic cancer, kidney cancer, bladder cancer, colon cancer, gastroesophageal adenocarcinoma or liver cancer.

7. (canceled)

8. The method of claim 2, wherein the inverse agonist of RORs is a compound of Formula (I), (II), (III), or (IV), a pharmaceutically acceptable salts thereof, or stereoisomers thereof: 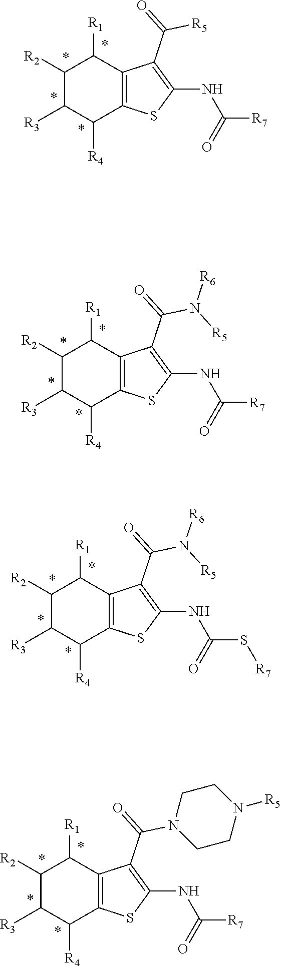
wherein,
R₁, R₂, R₃, and/or R₄ group(s) is/are H, halogen, NO₂, 1-6 alkoxy, OH, NH₂, 1-6 alkyl, 1-6 alkenyl, 1-6 haloalkyl, N-dialkyl, haloalkoxy, 1-6 hydroxyalkyl, and/or —CO₂ (1-6 alkyl);
R₅ is a substituted or unsubstituted five or six membered saturated or unsaturated heterocycle, aryl, alkylarene, halo aryl, ring substituted alkylarene, ring substituted alkylhexane, ring substituted alkylcyclopentane, haloaryl, benzene, phenyl, benzyl, pyridine, pyrimidine, pyridine, imidazole, diazole, triazole, thiadiazole, imidazolidine, thizolidine, pyrrolidine, piperazine, piperidine, pyridazine, pyrazine, triazine, 1H pyrrole, 2H pyrrole, pyrroline, pyrazolidine, pyrazoline, thiazole, isothiazole, isoxazole, haloalkyl, cyanoalkyl, methylpyrimidine, toluene, methylpyridine, methylimidazole, methyldiazole, methyltriazole, methylthiadiazole, methylimidazolidine, methylthizolidine, methylpyrrolidine, methylpiperazine, methylpiperidine, methylpyridazine, methylpyrazine, methyltriazine, methylpyrrole, methylpyrroline, methylpyrazolidine, methylpyrazoline, methylthiazole, methylisothiazole, methylisoxazole, or arylalkyl,
R₆ is H, 1-6 alkyl, or may form a five or six ring structure with R₅; and
R₇ is a substituted or unsubstituted five or six membered saturated or unsaturated heterocycle, ring substituted alkylarene, ring substituted alkylhexane, ring substituted alkylcyclopentane, substituted haloaryl, substituted benzene, substituted phenyl, benzyl, pyrimidine, pyridine, imidazole, diazole, triazole, thiadiazole, imidazolidine, thizolidine, pyrrolidine, piperazine, aryl, halo aryl, alkylarene, piperidine, pyridazine, pyrazine, triazine, 1H pyrrole, 2H pyrrole, pyrroline, pyrazolidine, pyrazoline, thiazole, isothiazole, isoxazole, cyanoalkyl, methylpyrimidine, toluene, methylpyridine, methylimidazole, methyldiazole, methyltriazole, methylthiadiazole, methylimidazolidine, methylthizolidine, methylpyrrolidine, methylpiperazine, methylpiperidine, methylpyridazine, methylpyrazine, methyltriazine, methylpyrrole, methylpyrroline, methylpyrazolidine, methylpyrazoline, methylthiazole, methylisothiazole, methylisoxazole, or arylalkyl.

9. (canceled)

10. The method of claim 2, wherein the inverse agonist of RORs is a compound or pharmaceutically acceptable salts thereof, or stereoisomers thereof selected from the group consisting of:
N-(3-(4-benzylpiperazine-1-carbonyl)-4,5,6,7-tetrahydro-benzo[b]thiophen-2-yl)-2-fluorobenzamide;
N-(3-(benzylcarbamoyl)-4,5,6,7-tetrahydrobenzo[b]thiophen-2-yl)-5-chloro-2-(methylthio)pyrimidine-4-carboxamide;
5-chloro-N-(3-{[(1,1-dioxidotetrahydro-3-thienyl)amino]-carbonyl}-6-methyl-4,5,6,7-tetrahydro-1-benzothien-2-yl)-2-(methylthio)-4-pyrimidine-carboxamide;
N-(6-ethyl-3-{[(2-methylphenyl)amino]carbonyl}-4,5,6,7-tetrahydro-1-benzothien-2-yl)-1-methyl-1H-pyrazole-5-carboxamide;
N-{6-tert-butyl-3-[(4-methyl-1-piperazinyl) carbonyl]-4,5,6,7-tetrahydro-1-benzothien-2-yl}-2-fluorobenzamide;
2-fluoro-N-{6-methyl-3-[(4-methyl-1-piperazinyl) carbonyl]-4,5,6,7-tetrahydro-1-benzothien-2-yl}benzamide;
N-benzyl-2-[(trifluoroacetyl)amino]-4,5,6,7-tetrahydro-1-benzothiophene-3-carboxamide;
N-benzyl-2-({[(4-methyl-2-pyrimidinyl)thio]acetyl}amino)-4,5,6,7-tetrahydro-1-benzothiophene-3-carboxamide;
N-benzyl-2-[(1-piperidinylacetyl)amino]-4,5,6,7-tetrahydro-1-benzothiophene-3-carboxamide;
N-benzyl-2-{[(4-methyl-1-piperazinyl) acetyl]amino}-4,5,6,7-tetrahydro-1-benzothiophene-3-carboxamide;
N-benzyl-2-{[(4-methyl-1-piperidinyl) acetyl]amino}-4,5,6,7-tetrahydro-1-benzothiophene-3-carboxamide;
N-(3-(2-methylpyrrolidine-1-carbonyl)-4,5,6,7-tetrahydro-benzo[b]thiophen-2-yl) nicotinamide;
N-(3-((2R,4R)-2,4-dimethylpiperidine-1-carbonyl)-4,5,6,7-tetrahydrobenzo[b]thiophen-2-yl) nicotinamide;
N-(3-(((1r,4r)-4-hydroxycyclohexyl) carbamoyl)-4,5,6,7-tetrahydrobenzo[b]thiophen-2-yl) nicotinamide;
N-(3-(3-ethylpyrrolidine-1-carbonyl)-4,5,6,7-tetrahydro-benzo[b]thiophen-2-yl) nicotinamide;
N-benzyl-2-(2-((1-methyl-1H-imidazol-2-yl)thio) acetamido)-4,5,6,7-tetrahydrobenzo[b]thiophene-3-carboxamide;
N-(3-(3-(hydroxymethyl) pyrrolidine-1-carbonyl)-4,5,6,7-tetrahydrobenzo[b]thiophen-2-yl) nicotinamide;
N-(6,6-dimethyl-3-(morpholine-4-carbonyl)-4,5,6,7-tetrahydrobenzo[b]thiophen-2-yl) pyrazine-2-carboxamide;
N-(3-(((1-methyl-1H-1,2,4-triazol-3-yl)methyl) carbamoyl)-4,5,6,7-tetrahydrobenzo[b]thiophen-2-yl) nicotinamide;
(S)—N-(3-((1-cyanoethyl) carbamoyl)-4,5,6,7-tetrahydro-benzo[b]thiophen-2-yl) nicotinamide;
N-(3-((piperidin-4-ylmethyl) carbamoyl)-4,5,6,7-tetrahydro-benzo[b]thiophen-2-yl) nicotinamide;
N-(3-benzoyl-4,5,6,7-tetrahydrobenzo[b]thiophen-2-yl) pyrazine-2-carboxamide;
N-(3-benzoyl-4,5,6,7-tetrahydrobenzo[b]thiophen-2-yl)-5-methyl-2-(methylsulfonyl)pyrimidine-4-carboxamide;
N-(4-ethylphenyl)-3-(hydroxymethyl)-N-isobutyl-4-((tetrahydro-2H-pyran-4-yl) methoxy)benzenesulfonamide;
(7S)-N-{[5-(Ethylsulfonyl)-2-pyridinyl]methyl}-7-isopropyl-6-{[trans-4-(trifluoromethyl)cyclohexyl]methyl}-6,7-dihydro-5H-pyrrolo[3,4-b]pyridine-3-carboxamide;
N-{3-[(3-methylbut-2-en-1-yl) {methyl[trans-4-(pyridin-4-yl)cyclohexyl]carbamoyl}amino]phenyl}benzamide;
2-(1-(2,4-dichloro-3-((7-chloro-5-(trifluoromethyl)-1H-indol-1-yl)methyl)benzoyl) piperidin-4-yl) acetic acid;
(S)-2-(4-cyclopropyl-6-methylpyrimidin-5-yl)-8-(1-cyclopropylethyl)-6-(((5-(methylsulfonyl)pyridin-2-yl)methyl)amino) pteridin-7 (8H)-one;
(S)-6-(2,6-dimethylpyrimidin-4-yl)-N-(4-(ethylsulfonyl)benzyl)-6-methyl-5-oxo-5,6,7,8-tetrahydroquinoline-2-carboxamide;
2-(2-((S)-(3,5-dimethylisoxazol-4-yl) (hydroxy)methyl)benzofuran-5-yl)-N—((S)-(2,4-dimethylphenyl) (phenyl)methyl) acetamide;
N-(4-(1,1,1,3,3,3-hexafluoro-2-hydroxypropan-2-yl)phenyl)-N-(2,2,2-trifluoroethyl)benzenesulfonamide;
1,1,1,3,3,3-hexafluoro-2-(2-fluoro-4′-((4-(pyridin-4-ylmethyl) piperazin-1-yl)methyl)-[1,1′-biphenyl]-4-yl) propan-2-ol;
(1R,3aS,5aR,5bR,7aR,9S, 11aR, 11bR, 13aR, 13bR)-N-(3-(dimethylamino) propyl)-9-hydroxy-5a,5b,8,8,11a-pentamethyl-1-(prop-1-en-2-yl) icosahydro-3aH-cyclopenta[a]chrysene-3a-carboxamide; and

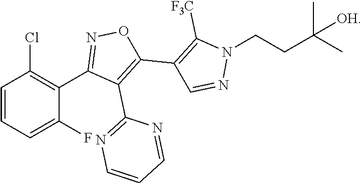 [SMILES: CC(C)(O)CCn1ncc(-c2onc(-c3c(F)cccc3Cl)c2-c2ncccn2)c1C(F)(F)F]

11. (canceled)

12. The method of claim 2, wherein the inverse agonist of RORs is a compound or pharmaceutically acceptable salts thereof, or stereoisomers thereof selected from the group consisting of: 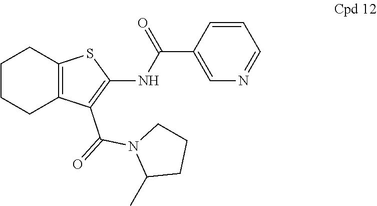 [SMILES: CC1CCCN1C(=O)c1c(NC(=O)c2cccnc2)sc2c1CCCC2]
N-(3-(2-methylpyrrolidine-1-carbonyl)-4,5,6,7-tetrahydrobenzo[b]thiophen-2-yl) nicotinamide 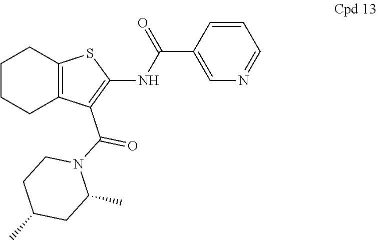 [SMILES: C[C@@H]1CCN(C(=O)c2c(NC(=O)c3cccnc3)sc3c2CCCC3)[C@H](C)C1]
N-(3-((2R,4R)-2,4-dimethylpiperidine-1-carbonyl)-4,5,6,7-tetrahydrobenzo[b]thiophen-2-yl) nicotinamide 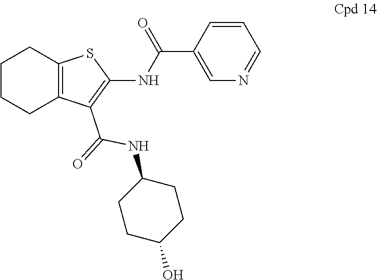 [SMILES: O=C(Nc1sc2c(c1C(=O)N[C@H]1CC[C@H](O)CC1)CCCC2)c1cccnc1]
N-(3-(((1r,4r)-4-hydroxycyclohexyl) carbamoyl)-4,5,6,7-tetrahydrobenzo[b]thiophen-2-yl) nicotinamide 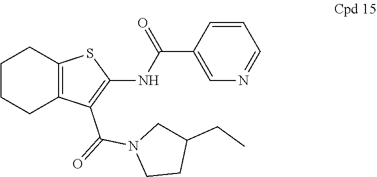 [SMILES: CCC1CCN(C(=O)c2c(NC(=O)c3cccnc3)sc3c2CCCC3)C1]
N-(3-(3-ethylpyrrolidine-1-carbonyl)-4,5,6,7-tetrahydrobenzo[b]thiophen-2-yl) nicotinamide 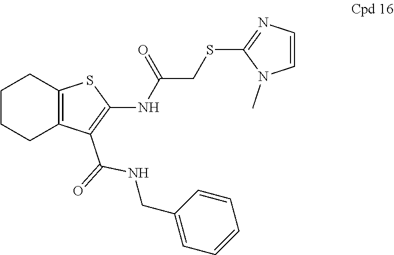 [SMILES: Cn1ccnc1SCC(=O)Nc1sc2c(c1C(=O)NCc1ccccc1)CCCC2]
N-benzyl-2-(2-((1-methyl-1H-imidazol-2-yl)thio) acetamido)-4,5,6,7-tetrahydrobenzo[b]thiophene-3-carboxamide 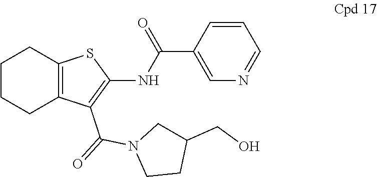 [SMILES: O=C(Nc1sc2c(c1C(=O)N1CCC(CO)C1)CCCC2)c1cccnc1]
N-(3-(3-(hydroxymethyl) pyrrolidine-1-carbonyl)-4,5,6,7-tetrahydrobenzo[b]thiophen-2-yl) nicotinamide 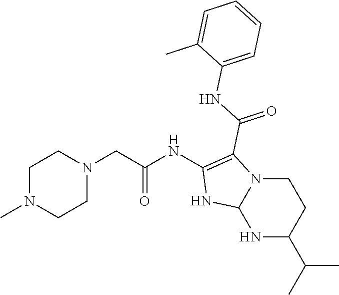 [SMILES: Cc1ccccc1NC(=O)C1=C(NC(=O)CN2CCN(C)CC2)NC2NC(C(C)C)CCN12]
7-isopropyl-2-(2-(4-methylpiperazin-1-yl) acetamido)-N-(o-tolyl)-1,5,6,7,8,8a-hexahydroimidazo[1,2-a]pyrimidine-3-carboxamide 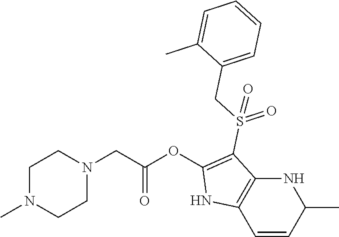
5-methyl-3-((2-methylbenzyl) sulfonyl)-4,5-dihydro-1H-pyrrolo[3,2-b]pyridin-2-yl 2-(4-methylpiperazin-1-yl)acetate 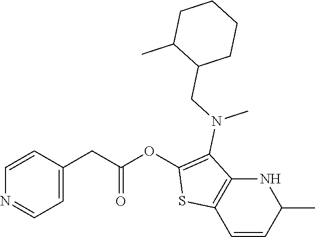 [SMILES: CC1C=Cc2sc(OC(=O)Cc3ccncc3)c(N(C)CC3CCCCC3C)c2N1]
5-methyl-3-(methyl((2-methylcyclohexyl)methyl)amino)-4,5-dihydrothieno[3,2-b]pyridin-2-yl 2-(pyridin-4-yl)acetate 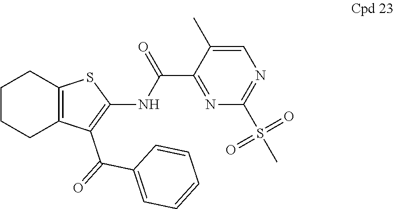 [SMILES: Cc1cnc(S(C)(=O)=O)nc1C(=O)Nc1sc2c(c1C(=O)c1ccccc1)CCCC2]
N-(3-benzoyl-4,5,6,7-tetrahydrobenzo[b]thiophen-2-yl)-5-methyl-2-(methylsulfonyl)pyrimidine-4-carboxamide 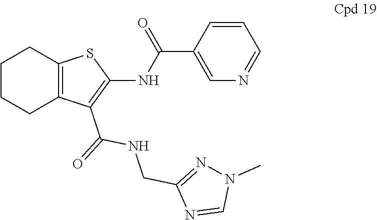 [SMILES: Cn1cnc(CNC(=O)c2c(NC(=O)c3cccnc3)sc3c2CCCC3)n1]
N-(3-(((1-methyl-1H-1,2,4-triazol-3-yl)methyl) carbamoyl)-4,5,6,7-tetrahydrobenzo[b]thiophen-2-yl) nicotinamide 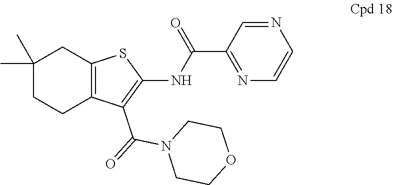 [SMILES: CC1(C)CCc2c(sc(NC(=O)c3cnccn3)c2C(=O)N2CCOCC2)C1]
N-(6,6-dimethyl-3-(morpholine-4-carbonyl)-4,5,6,7-tetrahydrobenzo[b]thiophen-2-yl) pyrazine-2-carboxamide

13-25. (canceled)

26. The method of claim 2, further comprising one or more additional compounds selected from the group consisting of:
(a) a cytotoxic agent; said cytotoxic agent being taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, Gemcitabine, Paclitaxel, FOLFIRINOX is a chemotheraphy combination to treat pancreatic cancer and is composed of leucovorin, folinic acid, flurouracil, irinotecan hydrochloride, oxaliplatin, analogs or homologs thereof, or a combination thereof,
(b) an antimetabolite; said antimetabolite being methotrexate, 6-mercaptopurine, 6-thioguanine, cytarabine, 5-fluorouracil decarbazine, or a combination thereof,
(c) an alkylating agent; said alkylating agent being mechlorethamine, thioepa chlorambucil, melphalan, carmustine (BSNU), lomustine (CCNU), cyclothosphamide, busulfan, dibromomannitol, streptozotocin, mitomycin C, cis-dichlorodiamine platinum (II) (DDP) cisplatin, or a combination thereof,
(d) an anthracycline; said anthracycline being daunorubicin, doxorubicin, or a combination thereof,
(e) an antibiotic; said antibiotic being dactinomycin, bleomycin, mithramycin, anthramycin (AMC), or a combination thereof,
(f) an anti-mitotic agent; said anti-mitotic agent being vincristine, vinblastine, or a combination thereof,
(g) an hormone therapy; said hormone therapy being a luteinizing hormone-releasing hormone (LHRH) antagonist,
(h) a signal transduction inhibitor; said signal transduction inhibitor being imatinib, trastuzumab, or a combination thereof,
(i) a gene expression modulator; said gene expression modulator being a siRNA, a shRNA, an antisense oligonucleotide, an HDAC inhibitor, or a combination thereof,
(j) an apoptosis inducer; said apoptosis inducer being a recombinant human TNF-related apoptosis-inducing ligand (TRAIL)
(k) an angiogenesis inhibitor, said angiogenesis inhibitor being sorafenib, sunitinib, pazopanib, everolimus or a combination thereof
(l) an immunotherapy agent, said immunotherapy agent being an Immune Checkpoint Inhibitors, a monoclonal antibody, a chimeric antigen receptors (CARs)-T-Cell, or a combination thereof,
(m) an Immune Checkpoint Inhibitor and
a pharmaceutically acceptable carrier.

27-39. (canceled)

40. A whole blood diagnostic test to monitor cancer patients response to treatment and help predict patient outcomes; which comprises measuring:
RORC+ neutrophils, total lymphocytes and ratio of ROC+ neutrophils versus total lymphocytes in a blood sample before and after treatment with a ROR inverse agonist according to the method of claim 2,
RORC+ neutrophils and FOXP3+ Treg lymphocytes and ratio of RORC+ neutrophils versus FOXP3+ Treg lymphocytes in a blood sample before and after treatment with a ROR inverse agonist according to the method of claim 2,
RORC+ neutrophils and CD8+ Treg lymphocytes and ratio of RORC+ neutrophils versus CD8+ Treg lymphocytes in a blood sample before and after treatment with a ROR inverse agonist according to the method of claim 2,
ratio of RORC+ neutrophils versus FOXP3+ Treg lymphocytes, and ratio of RORC+ neutrophils versus CD8+ Treg lymphocytes in a blood sample before and after treatment with a ROR inverse agonist according to the method of claim 2, or
ratio of RORC+ neutrophils, to ratio of FOXP3+ Treg lymphocytes to CD8+ Treg lymphocytes in a blood sample before and after treatment with a ROR inverse agonist according to the method of claim 2,
wherein an increase in the ratio is indicative of a poor response to the treatment.

41-44. (canceled)
